(19) United States
(12) Patent Application Publication
(10) Pub. No.: US 2006/0029544 A1
(43) Pub. Date: Feb. 9, 2006

(54) Receptor-binding cyclic peptides and methods of use

(75) Inventors: Julie Sutcliffe-Goulden, Woodland, CA (US); John Marshall, London (GB); Mark Howard, Hersden (GB); Michael O'Doherty, Chislehurst (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: The Regents of the University of California Office of Technology Transfer, Oakland, CA (US); Cancer Research Technology Ltd., London (GB); Enterprises of Kings College London Capital House, London (GB)

(21) Appl. No.: 11/198,884

(22) Filed: Aug. 5, 2005

Related U.S. Application Data
(60) Provisional application No. 60/599,846, filed on Aug. 6, 2004.

(51) Int. Cl. A61K 51/00; C07K 7/64

(52) U.S. Cl. 424/1.69; 530/317; 514/6

(57) ABSTRACT

The present invention provides novel receptor-binding cyclic peptides that advantageously display high receptor binding affinity and selectively. More particularly, the present invention provides integrin-binding cyclic peptides containing an integrin-binding motif such as an RGD motif, an aromatic amino acid such as a tyrosine residue, and a lysine residue having a pi-pi stacking moiety conjugated to its ε-amino group. Methods for identifying receptor-binding cyclic peptides and for using the cyclic peptides of the present invention for imaging a tumor, organ, or tissue and for treating cancer, inflammatory diseases, and autoimmune diseases are also provided.

### CROSS-REFERENCES TO RELATED APPLICATIONS

The present application claims priority to U.S. Provisional Application No. 60/599,846, filed Aug. 6, 2004, which is herein incorporated by reference in its entirety for all purposes.

### BACKGROUND OF THE INVENTION

Cell adhesion is a process by which cells associate with each other, migrate towards a specific target, or localize within the extracellular matrix. Cell adhesion constitutes one of the fundamental mechanisms underlying numerous biological phenomena. For example, cell adhesion is responsible for the adhesion of hematopoietic cells to endothelial cells and the subsequent migration of those hematopoietic cells out of blood vessels and to the site of injury. As such, cell adhesion plays a role in pathologies such as tumor metastasis, inflammation, and autoimmune disease in mammals.

Investigations into the molecular basis for cell adhesion have revealed that various cell surface macromolecules, collectively known as cell adhesion molecules or receptors, mediate cell-cell and cell-extracellular matrix interactions. For example, members of the integrin family of cell surface receptors mediate cell-cell and cell-extracellular matrix interactions and regulate cell motility, migration, survival, and proliferation (Hynes, Cell, 69:11-25 (1992); Hynes, Cell, 110:673-687 (2002)). Integrins are non-covalent heterodimeric glycoprotein complexes consisting of two subunits, α and β. To date, more than 18 different α subunits and more than 9 different β subunits have been identified in mammals. The extracellular globular domain of integrins associate with their ligands via short peptide motifs. The first of these ligand-recognition sites to be identified was the arginine, glycine, aspartic acid (RGD) motif, identified from the smallest active fragment of fibronectin. The RGD motif has also been found in many other extracellular matrix and serum proteins including vitronectin, laminin, fibrinogen, von Willebrand factor, and some collagens.

Integrins are essential in many biological processes including tissue development, platelet aggregation, and wound healing. Integrins are also implicated in a variety of diseases and disorders including cancer, inflammation, autoimmune diseases, and genetic-diseases. For example, α₅β₁, αᵥβ₃, and αᵥβ₅ integrins play critical roles in promoting tumor metastasis and angiogenesis (Hood and Cheresh, Nat. Rev. Cancer, 2:91-100 (2002); Jin and Varner, Brit. J Cancer, 90:561-565 (2004)). In particular, αᵥβ₃ integrin is implicated in promoting cell growth, inhibiting apoptosis, increasing protease production, promoting invasion of certain tumors, and promoting angiogenesis. Further, αᵥβ₃ integrin plays a critical role in activated macrophage-dependent inflammation, osteoclast-mediated bone resorption, and neovascularization, all of which are involved in pathologies such as rheumatoid arthritis and related arthropathies (Wilder, Ann. Rheum. Dis., 61(Suppl II):ii96-ii99 (2002)).

αᵥβ₃ integrin is expressed on a variety of cells including melanoma, glioblastoma, and osteoclasts and participates in a wide variety of both cell-cell and cell-matrix adhesive interactions. The expression of αᵥβ₃ integrin is upregulated on activated endothelial cells during angiogenesis. Further, αᵥβ₃ integrin is typically not expressed strongly in resting cells and tissues but is significantly increased in several tumors including cutaneous melanoma, glioblastoma, and Kaposi's sarcoma as well as at sites of inflammation. As with many of the integrins, αᵥβ₃ integrin binds its ligand via the RGD motif. αᵥβ₃ integrin ligands include, for example, vitronectin, fibronectin, fibrinogen, thrombospondin, osteopontin, von Willebrand factor, and proteolyzed collagen.

Given the vital role that αᵥβ₃ integrin plays in diseases and disorders such as tumor metastasis, angiogenesis, and inflammation, the notion of blocking its function to achieve therapeutic benefits has been explored. For example, intra-articular administration of an αᵥβ₃ integrin cyclic peptide antagonist to rabbits with antigen-induced arthritis inhibited synovial angiogenesis, inflammatory cell infiltration, and bone and cartilage destruction (Storgard et al., J. Clin. Invest., 103:47-54 (1999)). However, the cyclic peptide antagonist used in the study also exhibited activity against the closely related integrin, αᵥβ₅ integrin. As such, there is a need in the art for integrin-binding cyclic peptides having improved receptor binding affinity and selectively. Further, there is a need in the art for using integrin-binding cyclic peptides having improved receptor binding affinity and selectively for treating diseases or disorders such as inflammatory diseases, autoimmune diseases, or cancer. Moreover, there is a need in the art for using integrin-binding cyclic peptides having improved receptor binding affinity and selectively for imaging tumors, organs, or tissues in an individual. The present invention satisfies these and other needs.

### BRIEF SUMMARY OF THE INVENTION

The present invention provides novel receptor-binding cyclic peptides (e.g., antagonists) that advantageously display high receptor binding affinity and selectively. More particularly, the present invention provides integrin-binding cyclic peptides containing an integrin-binding motif such as an RGD motif, an aromatic amino acid such as a tyrosine residue, and a lysine residue having a pi-pi stacking moiety conjugated to its ε-amino group. Methods for identifying receptor-binding cyclic peptides and for using the cyclic peptides of the present invention for imaging a tumor, organ, or tissue and for treating cancer, inflammatory diseases, and autoimmune diseases are also provided.

As such, in one aspect, the present invention provides a cyclic peptide having the formula: 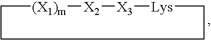
wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration.

In some embodiments, m is 0 or 1; X₂ is an integrin-binding motif; X₃ is Tyr, Tyr(Me), or Phe; the ε-amino group of Lys has a benzoyl group conjugated thereto; and X₃ and Lys have an L-configuration in the above formula. In preferred embodiments, the cyclic peptide has the following formula: 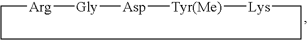
wherein
the ε-amino group of Lys has a 4-[¹⁸F]-fluorobenzoyl group or a 4-[¹⁹F]-fluorobenzoyl group conjugated thereto.

In another aspect, the present invention provides a method for imaging a tumor, organ, or tissue, the method comprising:
(a) administering to a subject in need of such imaging, a cyclic peptide having the formula: 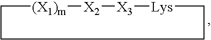
   wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration; and
(b) detecting the cyclic peptide to determine where the cyclic peptide is concentrated in the subject.

In yet another aspect, the present invention provides a method for treating cancer in a subject in need thereof, the method comprising:
administering to the subject a therapeutically effective amount of a cyclic peptide having the formula: 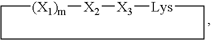
   wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration.

In still yet another aspect, the present invention provides a method for treating an inflammatory or autoimmune disease in a subject in need thereof, the method comprising:
administering to the subject a therapeutically effective amount of a cyclic peptide having the formula: 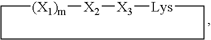
   wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration.

In a further aspect, the present invention provides a method for identifying a receptor-binding cyclic peptide, the method comprising:
(a) contacting a receptor or fragment thereof with a cyclic peptide having the formula: 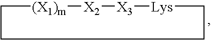
   wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration; and
(b) determining the binding of the cyclic peptide to the receptor or fragment thereof.

In additional aspects, the present invention provides a kit for imaging a tumor, organ, or tissue in a subject, for treating cancer in a subject in need thereof, or for treating an inflammatory or autoimmune disease in a subject in need thereof, the kit comprising:
(a) a container holding a cyclic peptide having the formula: 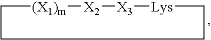
   wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration; and
(b) directions for use of the cyclic peptide in imaging a tumor, organ, or tissue, in treating cancer, or in treating an inflammatory or autoimmune disease.

Other objects, features, and advantages of the present invention will be apparent to one of skill in the art from the following detailed description and figures.

### BRIEF DESCRIPTION OF THE DRAWINGS

FIG. 1 shows the sequences of the peptides of the present invention, with resin attachment and side-chain protection. Abbreviations: Alloc, allyloxycarbonyl; Mtt, 4-methyltrityl; Pbf, 2,2,4,6,7-pentamethyldihydrobenzoftiran-5-sulphonyl; tBu, tert-butyl; PAL, 5-(4-(9-fluorenylmethoxycarbonyl)aminomethyl-3,5-dimethoxyphenoxy)-valeric acid.

FIG. 2 shows a diagram of ELISAs performed using αᵥβ₃-mFc (FIG. 2A), α_{5β}₁-hFc (FIG. 2B), α_{IIb}β₃-mFc (FIG. 2C), and αᵥβ₅-mFc (FIG. 2D).

FIG. 3 shows the percent binding of the vitronectin ligand to αᵥβ₅ integrin in the presence of linear (A), cyclic (B), or 4-[¹⁹F]-fluorobenzoyl cyclic (C) RGD peptides at concentrations of 2 μM, 20 μM, and 200 μM.

FIG. 4 shows the percent binding of the 50 kDa fibronectin ligand to α₅β₁, integrin in the presence of linear (A), cyclic (B), or 4-[¹⁹F]-fluorobenzoyl cyclic (C) RGD peptides at concentrations of 2 μM, 20 μM, and 200 μM.

FIG. 5 shows the percent binding of the fibrinogen ligand to α_{IIb}β₃ integrin in the presence of linear (A), cyclic (B), or 4-[¹⁹F]-fluorobenzoyl cyclic (C) RGD peptides at concentrations of 2 μM, 20 μM, and 200 μM.

FIG. 6 shows the percent binding of the 50 kDa fibronectin ligand to αᵥβ₃ integrin in the presence of linear (A), cyclic (B), or 4-[¹⁹F]-fluorobenzoyl cyclic (C) RGD peptides at concentrations of 2 μM, 20 μM, and 200 μM.

FIG. 7 shows the percent binding of the vitronectin ligand to αᵥβ₅ integrin in the presence of peptides C1, C3, C7, C9, and C10 at concentrations of 2 nM, 20 nM, 200 nM, and 2 μM.

FIG. 8 shows the percent binding of the 50 kDa fibronectin ligand to α₅β₁ integrin in the presence of peptides C1, C3, C7, C9, and C10 at concentrations of 2 nM, 20 nM, 200 nM, and 2 μM.

FIG. 9 shows the percent binding of the fibrinogen ligand to α_{IIb}β₃ integrin in the presence of peptides C1, C3, C7, C9, and C10 at concentrations of 2 nM, 20 nM, 200 nM, and 2 μM.

FIG. 10 shows the percent binding of the 50 kDa fibronectin ligand to αᵥβ₃ integrin in the presence of peptides C1, C3, C7, C9, and C10 at concentrations of 2 nM, 20 nM,200 nM, and 2 μM.

FIG. 11 shows titration curves of the inhibitory effects of peptide C7 on (A) αᵥβ₅; (B) α₅β₁; (C) α_{IIb}β₃; and (D) αᵥβ₃ integrin.

FIG. 12 shows titration curves of the inhibitory effects of peptide C10 on (A) αβ₅; (B) α₅β₁; (C) α_{IIb}β₃; and (D) αᵥβ₃ integrin.

FIG. 13 shows the effect of (A) A7, B7, and C7; and (B) A10, B10, and C10 on the binding of [⁵¹Cr]-VUP cells to vitronectin.

FIG. 14 shows the effect of (A) A7, B7, and C7; and (B) A10, B10, and C10 on the binding of [⁵¹Cr]-A375M cells to vitronectin.

FIG. 15 shows the effect of (A) A7, B7, and C7; and (B) A10, B10, and C10 on the binding of [⁵¹Cr]-VUP cells to laminin.

FIG. 16 shows the effect of (A) A7, B7, and C7; and (B) A10, B10, and C10 on the binding of [⁵¹Cr]-A375M cells to laminin.

FIG. 17 shows the fingerprint regions of the ¹H TOCSY NMR spectra of (A) B7 and (B) C7. (A) The large number of vertical peak strips indicates multiple conformations adopted by B7. (B) C7 adopts a single conformation (amino acids are indicated by arrows).

FIG. 18 shows the biodistribution of [¹⁸F]-C7 in the tumor, organs, and tissues after peptide injection.

FIG. 19 shows images obtained from an ECAT 951R PET scanner identifying distinct areas of [¹⁸F]-C7 uptake 30 minutes after injection in the lower region of the mouse (right image, arrow) that were absent in the negative control (left image). The images represent the coronal PET image fused with the transmission scan.

### DETAILED DESCRIPTION OF THE INVENTION

I. Definitions

As used herein, the following terms have the meanings ascribed to them unless specified otherwise.

The term “amino acid” refers to naturally-occurring α-amino acids and their stereoisomers, as well as unnatural amino acids and their stereoisomers. “Stereoisomers” of amino acids refers to mirror image isomers of the amino acids, such as amino acids having an L-configuration (L-amino acids) or amino acids having a D-configuration (D-amino acids). For example, a stereoisomer of a naturally-occurring amino acid refers to the mirror image isomer of the naturally-occurring amino acid, i.e., the D-amino acid. Amino acids may be referred to herein by either their commonly known three letter symbols or by the one-letter symbols recommended by the FUPAC-IUB Biochemical Nomenclature Commission. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine).

The term “X₃ and Lys have the same configuration” refers to a cyclic peptide of the present invention wherein both X₃ and Lys are L-amino acids or both X₃ and Lys are D-amino acids. Preferably, both X₃ and Lys are L-amino acids (i.e., have the L-configuration) in the cyclic peptides of the present invention.

The term “RGD peptide” refers to a linear or cyclic peptide of the present invention which contains at least one copy of the Arg-Gly-Asp integrin-binding motif. The term “RGD cyclic peptide” refers to a cyclic peptide of the present invention which contains at least one copy of the Arg-Gly-Asp integrin-binding motif.

The term “aromatic amino acid” refers to any naturally-occurring ε-amino acid containing an aromatic ring structure such as tyrosine (Tyr), phenylalanine (Phe), or tryptophan (Trp), as well as analogs thereof.

Suitable Tyr analogs for use in the present invention include, without limitation, O-methyltyrosine (Tyr(Me)); O-ethyltyrosine (Tyr(Et)); O-benzyltyrosine (Tyr(Bzl)); homotyrosine (HoTyr); C₁-C₄ alkyltyrosines such as 2-methyltyrosine (Tyr(2-Me)) or 3-methyltyrosine (Tyr(3-Me)); C₁-C₄ alkoxytyrosines such as 2-methoxytyrosine (Tyr(2-OMe)) or 3-methoxytyrosine (Tyr(3-OMe)); halotyrosines such as 2-fluorotyrosine (Tyr(2-F)), 2-chlorotyrosine (Tyr(2-Cl)), 2-bromotyrosine (Tyr(2-Br)), 2-iodotyrosine (Tyr(2-I)), 3-fluorotyrosine (Tyr(3 -F)), 3-chlorotyrosine (Tyr(3 -Cl)), 3 -bromotyrosine (Tyr(3-Br)), 3-iodotyrosine (Tyr(3-I)), 3,5-difluorotyrosine (Tyr(diF)), 3,5-dichlorotyrosine (Tyr(diCl)), 3,5-dibromotyrosine (Tyr(diBr)), or 3,5-diiodotyrosine (Tyr(diI)); C₁-C₄ haloalkyltyrosines such as 2-trifluoromethyltyrosine (Tyr(2-CF₃)) or 3-trifluoromethyltyrosine (Tyr(3-CF₃)); azidotyrosines such as 2-azidotyrosine (Tyr(2-N₃))or 3-azidotyrosine (Tyr(3-N₃)); aminotyrosines such as 2-aminotyrosine (Tyr(2-NH₂)) or 3-aminotyrosine (Tyr(3-NH₂)); nitrotyrosines such as 2-nitrotyrosine (Tyr(2-NO₂)) or 3-nitrotyrosine (Tyr(3-NO₂)); cyanotyrosines such as 2-cyanotyrosine (Tyr(2-CN)) or 3-cyanotyrosine (Tyr(3-CN); benzoyltyrosines such as 2-benzoyltyrosine or 3-benzoyltyrosine; and carboxytyrosines such as 2-carboxytyrosine (Tyr(2-COOH) or 3-carboxytyrosine (Tyr(3-COOH). Preferably the Tyr analog is Tyr(Me).

Suitable Phe analogs for use in the present invention include, without limitation, phenylglycine (Phg); homophenylalanine (HoPhe); diphenylalanines such as 3,3-diphenylalanine (Dpa); C₁-C₄ alkylphenylalanines such as 2-methylphenylalanine (Phe(2-Me)), 3-methylphenylalanine (Phe(3-Me)), 4-methylphenylalanine (Phe(4-Me)), or 4-ethylphenylalanine (Phe(4-Et)); C₁-C₄ alkoxyphenylalanines such as 2-methoxyphenylalanine (Phe(2-OMe)), 3-methoxyphenylalanine (Phe(3-OMe)), 4-methoxyphenylalanine (Phe(4-OMe)), 3,4-dimethoxyphenylalanine (Phe(3,4-di OMe)), 4-ethoxyphenylalanine (Phe(4-OEt)), or 4-butoxyphenylalanine (Phe(4-OBu)); halophenylalanines such as 2-fluorophenylalanine (Phe(2-F)), 3-fluorophenylalanine (Phe(3-F)), 4-fluorophenylalanine (Phe(4-F)), 2-chlorophenylalanine (Phe(2-Cl)), 3-chlorophenylalanine (Phe(3-Cl)), 4-chlorophenylalanine (Phe(4-Cl)), 2-bromophenylalanine (Phe(2-Br)), 3-bromophenylalanine (Phe(3-Br)), 4-bromophenylalanine (Phe(4-Br)), 2-iodophenylalanine (Phe(2-I)), 3-iodophenylalanine (Phe(3-I)), 4-iodophenylalanine (Phe(4-I)), 3,4-difluorophenylalanine (Phe(3,4-di F)), 3,5-difluorophenylalanine (Phe(3,5-di F)), 2,4-dichlorophenylalanine (Phe(2,4-di Cl)), 3,4-dichlorophenylalanine (Phe(3,4-di Cl)), 2,3,4,5,6-pentafluorophenylalanine (Phe(F₅)), or 3,4,5-trifluorophenylalanine (Phe(F₃)); C₁-C₄ haloalkylphenylalanines such as 2-trifluoromethylphenylalanine (Phe(2-CF₃)), 3-trifluoromethylphenylalanine (Phe(3-CF₃)), or 4-trifluoromethylphenylalanine (Phe(4-CF₃)); azidophenylalanines such as 4-azidophenylalanine (Phe(4-N₃)); aminophenylalanines such as 4-aminophenylalanine (Phe(4-NH₂)); nitrophenylalanines such as 2-nitrophenylalanine (Phe(2-NO₂)), 3-nitrophenylalanine (Phe(3-NO₂)), or 4-nitrophenylalanine (Phe(4-NO₂)); cyanophenylalanines such as 2-cyanophenylalanine (Phe(2-CN)), 3-cyanophenylalanine (Phe(3-CN)), or 4-cyanophenylalanine (Phe(4-CN)); benzoylphenylalanines such as 4-benzoylphenylalanine (Bpa); carboxyphenylalanines such as 4-carboxyphenylalanine (Phe(4-COOH)); and halophenylglycines such as 2-fluorophenylglycine (Phg(2-F)), 3-fluorophenylglycine (Phg(3-F)), 4-fluorophenylglycine (Phg(4-F)), 2-chlorophenylglycine (Phg(2-Cl)), 3-chlorophenylglycine (Phg(3-Cl)), 4-chlorophenylglycine (Phg(4-Cl)), 2-bromophenylglycine (Phg(2-Br)), 3-bromophenylglycine (Phg(3-Br)), or 4-bromophenylglycine (Phg(4-Br)).

Suitable Trp analogs for use in the present invention include, without limitation, C₁-C₄ alkyltryptophans, C₁-C₄ alkoxytryptophans, halotryptophans, C₁-C₄ haloalkyltryptophans, azidotryptophans, aminotryptophans, nitrotryptophans, cyanotryptophans, benzoyltryptophans, and carboxytryptophans.

With respect to amino acid sequences, one of skill will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins, 1984).

The term “peptide” refers to a compound made up of a single chain of D- or L-amino acids or a mixture of D- and L-amino acids joined by peptide bonds. Generally, peptides of the present invention are from about 2 to about 50 amino acids in length. Preferably, the peptides of the present invention are from 4 to 25 amino acids in length, more preferably from 5 to 10 amino acids in length, and most preferably 5 or 6 amino acids in length. A “cyclic peptide” as used herein refers to a peptide in which the amino-terminus of the peptide or a side-chain on the peptide having a free amino group (e.g., lysine) is joined by a peptide bond to the carboxyl-terminus of the peptide or a side-chain on the peptide having a free carboxyl group (e.g., aspartic acid, glutamic acid). However, one skilled in the art will appreciate that heterodetic cyclic peptides formed by disulfide, ester, or ether bonds are also within the scope of the present invention.

The term “receptor-binding motif” as used herein refers to a sequence found in a peptide, polypeptide, or protein that is the recognition site for one or more receptors. In certain instances, the receptor-binding motif is found in a naturally-occurring peptide, polypeptide, or protein such as a ligand, co-receptor, adaptor molecule, signaling molecule, etc. In certain other instances, the receptor-binding motif is found in a synthetic or recombinant peptide, polypeptide, or protein. Typically, the receptor-binding motif comprises a short peptide sequence of from about 2 to about 25 amino acids in length, e.g., 2, 3, 4, 5, 6, 7, 8, 9, 10, 15, 20, or 25 amino acids in length. However, receptor-binding motifs greater than about 25 amino acids in length are also with the scope of the present invention. Suitable receptor-binding motifs for use in the present invention are described below.

The term “pi-pi stacking moiety” refers to an aromatic group that can participate in non-covalent aromatic-aromatic interactions (e.g., pi-pi stacking interactions) with one or more aromatic amino acid side-chains. Typically, the pi-pi stacking moiety interacts with the aromatic side-chain in a parallel displaced orientation. However, one skilled in the art will appreciate that other types of aromatic-aromatic interactions between the pi-pi stacking moiety and the aromatic side-chain including, for example, edge-face interactions (i.e., T-shaped orientations) are also within the scope of the present invention. Suitable pi-pi stacking moieties for use in the present invention include, without limitation, a benzoyl group, a benzyl group, a naphthoyl group, and a naphthyl group. Preferably, the pi-pi stacking moiety in the cyclic peptides of the present invention is a benzoyl group. Without being bound to any particular theory, the pi-pi stacking interaction between the pi-pi stacking moiety and the aromatic side-chain restricts (i.e., locks) the cyclic peptides of the present invention in a single conformation, thereby increasing their receptor affinity and selectively.

The term “therapeutically effective amount” refers to the amount of a cyclic peptide or a combination of cyclic peptides of the present invention that is capable of achieving a therapeutic effect in a subject in need thereof. For example, a therapeutically effective amount of a cyclic peptide or a combination of cyclic peptides can be the amount that is capable of preventing or relieving one or more symptoms associated with cancer, an inflammatory disease, or an autoimmune disease.

The term “cancer” refers to any of various malignant neoplasms characterized by the proliferation of anaplastic cells that tend to invade surrounding tissue and metastasize to new body sites. Examples of different types of cancer suitable for treatment using the present invention include, but are not limited to, lung cancer, breast cancer, bladder cancer, thyroid cancer, liver cancer, pleural cancer, pancreatic cancer, ovarian cancer, cervical cancer, testicular cancer, prostate cancer, colon cancer, anal cancer, bile duct cancer, gastrointestinal carcinoid tumors, esophageal cancer, oral cancer, gall bladder cancer, rectal cancer, appendix cancer, small intestine cancer, stomach (gastric) cancer, renal cancer, cancer of the central nervous system, skin cancer, choriocarcinomas; head and neck cancers, blood cancers, sarcomas (e.g., Kaposi's sarcoma), osteogenic sarcomas, fibrosarcoma, neuroblastoma, glioblastoma, melanoma (e.g., cutaneous melanoma), and lymphomas or leukemias such as B-cell lymphoma, non-Hodgkin's lymphoma, Burkitt's lymphoma, acute lymphoblastic leukemia, chronic lymphoid leukemia, monocytic leukemia, myelogenous leukemia, acute myelocytic leukemia, diffuse large B-cell lymphoma, follicle center lymphoma, Hodgkin's lymphoma, mantle cell lymphoma, marginal zone lymphoma, Waldenstrom's macroglobulinaemia, myeloma, monoclonal gammopathy of uncertain significance, large granular lymphocyte leukemia, T-prolymphocytic leukemia, Sezary Syndrome, common angio-immunoblastic and anaplastic large cell lymphomas, mycosis fingoides, lymphomatoid papulosis, small intestinal lymphoma, myelodysplastic syndrome, myeloproliferative disorders, paroxysmal nocturnal haemoglobinuria, and aplastic anemia. Preferably, the cyclic peptides of the present invention are used for treating cutaneous melanoma, glioblastoma, Kaposi's sarcoma, breast cancer, prostate cancer, or oral cancer.

The term “inflammatory disease” refers to a disease or disorder characterized or caused by inflammation. “Inflammation” refers to a local response to cellular injury that is marked by capillary dilatation, leukocytic infiltration, redness, heat, and pain that serves as a mechanism initiating the elimination of noxious agents and of damaged tissue. The site of inflammation can include, without limitation, the lungs, the pleura, a tendon, a lymph node or gland, the uvula, the vagina, the brain, the spinal cord, nasal and pharyngeal mucous membranes, a muscle, the skin, bone or bony tissue, a joint, the urinary bladder, the retina, the cervix of the uterus, the canthus, the intestinal tract, the vertebrae, the rectum, the anus, a bursa, and a follicle. Examples of different types of inflammatory diseases suitable for treatment using the present invention include, but are not limited to, inflammatory bowel disease (IBD), arthritis (e.g., rheumatoid arthritis), fibrositis, pelvic inflammatory disease, acne, psoriasis, actinomycosis, dysentery, biliary cirrhosis, Lyme disease, heat rash, Stevens-Johnson syndrome, systemic lupus erythematosus, mumps, autoimmune hepatitis, pemphigus vulgaris, and blastomycosis. Inflammatory bowel diseases are chronic inflammatory diseases of the gastrointestinal tract which include, without limitation, Crohn's disease (CD), ulcerative colitis (UC), and indeterminate colitis. Arthritis is an inflammatory condition that affects joints which includes, without limitation, acute arthritis, acute gouty arthritis, bacterial arthritis, chronic inflammatory arthritis, degenerative arthritis (osteoarthritis), infectious arthritis, juvenile arthritis, mycotic arthritis, neuropathic arthritis, polyarthritis, proliferative arthritis, psoriatic arthritis, juvenile rheumatoid arthritis, rheumatoid arthritis, venereal arthritis, and viral arthritis. Preferably, the cyclic peptides of the present invention are used for treating rheumatoid arthritis.

The term “autoimmune disease” refers to a disease or disorder resulting from an immune response against a self tissue or tissue component and includes a self antibody response or cell-mediated response. The term encompasses organ-specific autoimmune diseases, in which an autoimmune response is directed against a single tissue. Examples of different types of organ-specific autoimmune diseases suitable for treatment using the present invention include, but are not limited to, Type I diabetes mellitus, myasthenia gravis, vitiligo, Graves' disease, Hashimoto's disease, Addison's disease, autoimmune gastritis, and autoimmune hepatitis. The term also encompasses non-organ specific autoimmune diseases, in which an autoimmune response is directed against a component present in several or many organs throughout the body. Examples of different types of non-organ specific autoimmune diseases suitable for treatment using the present invention include, but are not limited to, systemic lupus erythematosus, progressive systemic sclerosis and variants, polymyositis, and dermatomyositis. Additional autoimmune diseases suitable for treatment using the present invention include, but are not limited to, pernicious anemia, primary biliary cirrhosis, autoimmune thrombocytopenia, Sjogren's syndrome, and multiple sclerosis.

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to a subject. Adminsitration is by any route, including parenteral, transdermal, and transmucosal (e.g., sublingual, buccal, gingival, palatal, nasal, vaginal, or rectal). Parenteral administration includes, e.g., intravenous, intramuscular, intra-arteriole, intradermal, subcutaneous, intraperitoneal, intratracheal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “subject” refers to any mammal suitable for imaging or therapy with the cyclic peptides of the present invention. Preferably, the subject is a human. However, one skilled in the art will appreciate that the subject can also be an animal such as a mouse, rat, dog, cat, hamster, guinea pig, livestock, and the like.

The term “nuclide” refers to a type of atom specified by its atomic number, atomic mass, and energy state, such as carbon 13 (¹³C). A “radionuclide” refers to a nuclide that exhibits radioactivity, such as carbon 14 (¹⁴C). “Radioactivity” refers to the radiation, including alpha particles, beta particles, nucleons, electrons, positrons, neutrinos, and gamma rays, emitted by a radioactive substance. Radionuclides suitable for use in the present invention include, but are not limited to, carbon 11 (¹¹C), nitrogen 13 (¹³N), oxygen 15 (¹⁵O), fluorine 18 (¹⁸F), phosphorus 32 (³²p), scandium 47 (⁴⁷Sc), cobalt 55 (⁵⁵Co), copper 60 (⁶⁰Cu), copper 61 (⁶¹ Cu), copper 62 (⁶²Cu), copper 64 (⁶⁴Cu), gallium 66 (⁶⁶Ga), copper 67 (⁶⁷Cu), gallium 67 (⁶⁷Ga), gallium 68 (⁶⁸Ga), rubidium 82 (⁸²Rb), yttrium 86 (¹⁶y), yttrium 87 (⁸⁷y), strontium 89 (⁸⁹Sr), yttrium 90 (⁹⁰Y), rhodium 105 (¹⁰⁵Rh), silver 111 (¹¹¹Ag), indium 111 (¹¹¹In), iodine 124 (¹²⁴I), iodine 125 (¹²⁵I), iodine 131 (¹³¹I), tin 117m (^{117m}Sn), technetium 99m (^{99m}Tc), promethium 149 (¹⁴⁹Pm), samarium 153 (¹⁵³Sm), holmium 166 (¹⁶⁶Ho), lutetium 177 (¹⁷⁷Lu), rhenium 186 (¹⁸⁶Re), rhenium 188 (¹⁸⁸Re), thallium 201 (²⁰¹T1), astatine 211 (²¹¹At), and bismuth 212 (²¹²Bi). As used herein, the “m” in ^{117m}Sn and ^{99m}Tc stands for meta state. Additionally, naturally occurring radioactive elements such as uranium, radium, and thorium, which typically represent mixtures of radioisotopes, are suitable examples of radionuclides. Preferably, the pi-pi stacking moiety is labeled with a nuclide such as ¹⁹F or a radionuclide such as ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, and ¹³¹I.

II. General

The present invention provides novel receptor-binding cyclic peptides (e.g., antagonists) that advantageously display high receptor binding affinity and selectively. More particularly, the present invention provides integrin-binding cyclic peptides containing an integrin-binding motif such as an RGD motif, an aromatic amino acid such as a tyrosine residue, and a lysine residue having a pi-pi stacking moiety conjugated to its ε-amino group. Methods for identifying receptor-binding cyclic peptides and for using the cyclic peptides of the present invention for imaging a tumor, organ, or tissue and for treating cancer, inflammatory diseases, and autoimmune diseases are also provided.

The present invention is based upon the surprising discovery that the pi-pi stacking interaction between the pi-pi stacking moiety and the aromatic side-chain restricts (i.e., locks) the cyclic peptides of the present invention in a single conformation, thereby increasing their receptor affinity and selectively. For example, the remarkable ability of the cyclic peptide C7 (see, Example 2 below) to adopt a single conformation is provided by a pi-pi stacking interaction between the benzoyl moiety conjugated to lysine and the aromatic side chain of tyrosine. As a result, the pi-pi stacking interaction locks C7 in a single conformation, thereby increasing its affinity and selectively for αᵥβ₃ integrin. As such, C7 is suitable for use as an imaging agent for imaging a tumor, organ, or tissue. C7 is also suitable for use as a therapeutic agent for treating cancer, an inflammatory disease, or an autoimmune disease.

This structural locking mechanism can also be used to restrict the conformation of other receptor-binding motifs into a more restrained structure that binds the target receptor with increased affinity and selectivity. Examples of suitable receptor-binding motifs include, without limitation, other integrin-binding motifs, growth factor receptor-binding motifs, cytokine receptor-binding motifs, TGF-β receptor-binding motifs, TNF-α receptor-binding motifs, G-protein coupled receptor-binding motifs, scavenger receptor-binding motifs, lipoprotein receptor-binding motifs, other immune cell receptor-binding motifs, and combinations thereof. As such, the conformational rigidity provided by the structural locking mechanism of the present invention produces receptor-binding cyclic peptides with improved target affinity and selectivity.

III. Description of the Embodiments

In one aspect, the present invention provides a cyclic peptide having the formula: 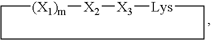
wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration.

In one embodiment, m is 0 or 1. For example, m is 0 when the cyclic peptide is a pentapeptide and X₂ is a receptor-binding motif having a three amino acid sequence such as an Arg-Gly-Asp (RGD) motif. Alternatively, m is 1 when the cyclic peptide is a hexapeptide and X₂ is a receptor-binding motif having a three amino acid sequence such as an RGD motif. In another embodiment, m is 2, 3, 4, 5, 6, 7, 8, 9, or 10. In some embodiments, X₁ comprises m amino acids that are independently selected from the group consisting of naturally-occurring amino acids or stereoisomers thereof; unnatural amino acids such as amino acid analogs, amino acid mimetics, synthetic amino acids, N-substituted glycines, and N-methyl amino acids; and combinations thereof.

In another embodiment, the pi-pi stacking moiety is selected from the group consisting of a benzoyl group, a benzyl group, a naphthoyl group, and a naphthyl group. Preferably, the pi-pi stacking moiety is a benzoyl group. In certain instances, the pi-pi stacking moiety is labeled with a nuclide. Suitable nuclides for use in labeling the pi-pi stacking moiety include, without limitation, ¹⁹F. For example, in the methods of the present invention, the cyclic peptide can have conjugated thereto a labeled pi-pi stacking moiety such as a 4-[¹⁹F]-fluorobenzoyl group, and the resulting labeled cyclic peptide can be used in, e.g., NMR spectroscopy. In certain other instances, the pi-pi stacking moiety is labeled with a radionuclide. Suitable radionuclides for use in labeling the pi-pi stacking moiety include, without limitation, ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, and ¹³¹I. For example, in the methods of the present invention, the cyclic peptide can have conjugated thereto a radiolabeled pi-pi stacking moiety such as a 4-[¹⁸F]-fluorobenzoyl group, and the resulting radiolabeled cyclic peptide can be used in, e.g., imaging a tumor, organ, or tissue or for treating a disease or disorder such as cancer, an inflammatory disease, or an autoimmune disease. Methods for the synthesis of a labeled pi-pi stacking moiety such as a 4-[¹⁸F]-fluorobenzoyl group and methods for their site-specific conjugation to peptides are described, e.g., in Example 1 below and in Sutcliffe-Goulden et al., Bioorg. & Med. Chem. Lett., 10:1501-1503 (2000) and Sutcliffe-Goulden et al., Eur. J. Nucl. Med., 29:754-759 (2002).

In yet another embodiment, X₃ is an aromatic amino acid selected from the group consisting of tyrosine (Tyr), phenylalanine (Phe), tryptophan (Trp), and an analog thereof. Suitable Tyr, Phe, and Trp analogs are described above. Preferably, the aromatic amino acid is Tyr, a Tyr analog such as Tyr(Me), or Phe.

Suitable receptor-binding motifs for use in the present invention include, without limitation, an integrin-binding motif, a growth factor receptor-binding motif, a cytokine receptor-binding motif, a transforming growth factor (TGF) receptor-binding motif, a tumor necrosis factor (TNF) receptor-binding motif, a G-protein coupled receptor-binding motif, a scavenger receptor-binding motif, a lipoprotein receptor-binding motif, other immune cell receptor-binding motifs, and combinations thereof. Preferably, the receptor-binding motif is an integrin-binding motif such as the RGD motif. Non-limiting examples of integrins that bind via the RGD motif include αᵥβ₁, αᵥβ₃, αᵥβ₅, αᵥβ₆, α_{IIb}β₃, α₃β₁, α₅β₁, and α₈β₁. Other integrin-binding motifs within the scope of the present invention include, without limitation, α₄β₁ integrin-binding motifs such as QIDS, ILDV, and LDI (see, e.g., Park et al., Lett. Pept. Sci., 8:171-178 (2002)); αᵥβ₆ integrin-binding motifs containing a DLXXL consensus sequence, e.g., RTDLDSLRTYTL (see, e.g., Kraft et al., J. Biol. Chem., 274:1979-1985 (1999)); α₂β₁ integrin-binding motifs such as DGEA; α_{IIb}β₃ integrin-binding motifs such as KQAGDV; α₂β₂ integrin-binding motifs such as GPRP; and α₄β₇ integrin-binding motifs such as EILDV. Additional examples of receptor-binding motifs include, without limitation, a cytokine receptor-binding motif such as the ELR sequence motif, which is observed in a variety of chemokines; and a scavenger receptor-binding motif such as the CSVTCG sequence motif, which is found in thrombospondin-1. One skilled in the art will know of additional integrin-binding motifs as well as other receptor-binding motifs that are suitable for use in the cyclic peptides of the present invention.

In certain instances, the receptor-binding motif comprises a peptide sequence found within a domain involved in ligand-receptor interactions. Examples of such domains include, without limitation, an epidermal growth factor (EGF) domain, a coiled-coil domain, a leucine rich repeat (LRR), an immunoglobulin (Ig) domain, a fibronectin domain, a laminin domain, a thrombospondin domain, a sterile alpha motif (SAM) domain, a meprin/A5-protein/PTPmu (MAM) domain, a postsynaptic density-95/Discs large/zona occludens-1 (PDZ) domain, and the like. One skilled in the art will appreciate that the region of the domain used as a receptor-binding motif can comprise the entire domain or a fragment thereof, such as, for example, 2, 3, 4, 5, 6, 7, 8, 9, 10, 15, 20, or 25 consecutive amino acids within the domain.

In a preferred embodiment, X₃ and Lys in the cyclic peptide have the same configuration (i.e., both are L-amino acids or D-amino acids). In certain instances, X₃ and Lys have an L-configuration (i.e., both are L-amino acids). In certain other instances, X₃ and Lys have a D-configuration (i.e., both are D-amino acids). In another preferred embodiment, X₂, X₃, and Lys have the same configuration. In certain instances, X₂, X₃, and Lys have an L-configuration. In certain other instances, X₂, X₃, and Lys have a D-configuration. Alternatively, X₃ and Lys have the same configuration and X₂ has a different configuration. In certain instances, X₃ and Lys have an L-configuration and X₂ has a D-configuration. In certain other instances, X₃ and Lys have a D-configuration and X₂ has an L-configuration. However, one skilled in the art appreciates that, as long as X₃ and Lys in the cyclic peptides of the present invention have the same configuration, the amino acids that make up X₁ or X₂ can be independently selected L-amino acids or D-amino acids. Further, one skilled in the art appreciates that D-amino acids and/or unnatural amino acids can be included in the cyclic peptides of the present invention to make them more resistant to cleavage or degradation from proteases found, for example, in plasma, the gastrointestinal tract, and/or tumor cells.

In another preferred embodiment, X₂ is an integrin-binding motif; X₃ is Tyr, Tyr(Me), or Phe; the i-amino group of Lys has a benzoyl group conjugated thereto; and X₃ and Lys have an L-configuration. Preferably, the integrin-binding motif has the amino acid sequence Arg-Gly-Asp (RGD) or Asp-Leu-X-X-Leu (DLXXL), where X is any amino acid. In certain instances, the benzoyl group is labeled with a nuclide. Suitable nuclides for use in labeling the benzoyl group include, without limitation, ¹⁹F. In certain other instances, the benzoyl group is labeled with a radionuclide. Suitable radionuclides for use in labeling the benzoyl group include, without limitation, ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, and ¹³¹I.

In a particularly preferred embodiment, the cyclic peptide has the following formula: 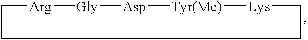
wherein the ε-amino group of Lys has a 4-[¹⁸F]-fluorobenzoyl group or a 4-[¹⁹F]-fluorobenzoyl group conjugated thereto. In certain instances, the ε-amino group of Lys has a 4-[¹⁸F]-fluorobenzoyl group conjugated thereto and the cyclic peptide has the amino acid sequence 4-[¹⁸F]-fluorobenzoyl cyclic (RGDY(OMe)K). Such radiolabeled cyclic peptides can be used in, e.g., imaging a tumor, organ, or tissue or for treating a disease or disorder such as cancer, an inflammatory disease, or an autoimmune disease. In certain other instances, the ε-amino group of Lys has a 4-[¹⁹F]-fluorobenzoyl group conjugated thereto and the cyclic peptide has the amino acid sequence 4-[¹⁹F]-fluorobenzoyl cyclic (RGDY(OMe)K). In a preferred embodiment, the cyclic peptide adopts a single conformation. In another preferred embodiment, the pi-pi stacking interaction between the fluorobenzoyl group and the aromatic Tyr side-chain restricts (i.e., locks) the cyclic peptide in a single conformation, thereby increasing its affinity and selectively for the αᵥβ₃ integrin receptor.

In another aspect, the present invention provides a method for imaging a tumor, organ, or tissue, the method comprising:
(a) administering to a subject in need of such imaging, a cyclic peptide having the formula: 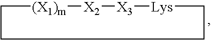
   wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration; and
(b) detecting the cyclic peptide to determine where the cyclic peptide is concentrated in the subject.

In one embodiment, m is 0 or 1. In another embodiment, m is 2, 3, 4, 5, 6, 7, 8, 9, or 10. In some embodiments, X₁ comprises m amino acids that are independently selected from the group consisting of naturally-occurring amino acids or stereoisomers thereof, unnatural amino acids, and combinations thereof.

In another embodiment, the pi-pi stacking moiety is benzoyl group, a benzyl group, a naphthoyl group, or a naphthyl group. Typically, the pi-pi stacking moiety is labeled with an imaging moiety such as a nuclide, a radionuclide, a chelating agent, a fluorophore, an antibody, and biotin or a derivative thereof. For example, the cyclic peptide can have conjugated thereto a radiolabeled pi-pi stacking moiety such as a 4-[¹⁸F]-fluorobenzoyl group, and the resulting radiolabeled cyclic peptide can be used in imaging a tumor, organ, or tissue using any radioimaging technique known in the art (e.g., PET imaging). One of ordinary skill in the art will appreciate other imaging moieties suitable for labeling the cyclic peptides of the present invention.

Generally, the nuclide or radionuclide can be attached directly to the pi-pi stacking moiety, or alternatively, the nuclide or radionuclide can be bound to a chelating agent attached to the pi-pi stacking moiety. Suitable radionuclides for direct conjugation in imaging a tumor, organ, or tissue include, without limitation, ¹¹C, ¹³N, ¹⁵O, ¹⁸F, ¹²⁴I, and ¹³¹I. Suitable radionuclides for use with a chelating agent in imaging a tumor, organ, or tissue include, without limitation, ⁵⁵Co, ⁶⁰Cu, ⁶¹Cu, ⁶²Cu, ⁶⁴Cu, ⁶⁶Ga, ⁶⁷Ga, ⁶⁸Ga, ⁸²Rb, ⁸⁶Y, ⁸⁷Y, ⁹⁰Y, ¹¹¹In, ^{99m}Tc, ²⁰¹Tl, and mixtures thereof. Suitable chelating agents include, but are not limited to, 1,4,7,10-tetraazacyclododecane-N,N′,N″,N′″-tetraacetic acid (DOTA), a bromoacetamidobenzyl derivative of DOTA (BAD), 1,4,8,11-tetraazacyclotetradecane-N,N′,N″,N′″-tetraacetic acid (TETA), diethylenetriaminepentaacetic acid (DTPA), EDTA, NTA, HDTA, their phosphonate analogs, and mixtures thereof. One of ordinary skill in the art will know of methods for labeling a pi-pi stacking moiety by attaching a nuclide, radionuclide, or chelating agent thereto and methods for their site-specific conjugation to peptides (see, e.g., Example 1 below).

Any device or method known in the art for detecting the radioactive emissions of radionuclides in a subject is suitable for use in the present invention for imaging a tumor, organ, or tissue. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tumors, tissues, organs, or body systems using a scintillation gamma camera, may be used for detecting the radiation emitted from a radiolabeled cyclic peptide of the present invention.

Preferably, positron emission tomography (PET), also called PET imaging or a PET scan, is used for detecting the radiation emitted from a radiolabeled cyclic peptide in a subject. PET is a non-invasive imaging technique that is assuming a rapidly increasing role in assisting clinicians in diagnosis and disease management. PET requires that a small molecule (e.g., a peptide) tagged with a positron emitting radionuclide is selectively retained in a tumor, tissue, or organ due to the local presence of a specific receptor (e.g., integrin receptor) or biological process (e.g., hypoxia or glucose metabolism). The positron emitting radionuclide generates two high-energy photons, which emerge from the body and are detected by the PET scanner. Computer analysis allows reconstruction in three-dimensions, thereby providing a detailed intra-corporeal location of the radioactivity. In certain instances, PET is used in differentiating between benign and malignant tumors, detecting and staging tumors, planning tumor treatment, monitoring tumor progression, evaluating tumor response to therapy, or imaging suspected tumor recurrence.

U.S. Pat. No. 5,429,133 describes a laparoscopic probe for detecting radiation concentrated in solid tissue tumors. Miniature and flexible radiation detectors intended for medical use are produced by Intra-Medical LLC, Santa Monica, Calif. Magnetic Resonance Imaging (MRI) or any other imaging technique known to one of skill in the art is also suitable for detecting the radioactive emissions of radionuclides. In addition, Computed Tomography (CT) scanning can be used to determine where the cyclic peptide is located in a subject. In instances where the imaging is performed on a small animal, high resolution PET scanners such as microPET or microPET II can be used (see, e.g., Cherry et al., IEEE Trans. Nucl. Sci., 44:1161-1166 (1997); Cherry, Phys. Med. Biol., 49:R13-48 (2004)). Furthermore, ultrasound imaging with air- or gas-filled contrast agents can be used to determine where the cyclic peptide is located in a subject (see, e.g., Bloch et al., IEEE Eng. Med. Biol. Mag., 23:18-29 (2004)). Regardless of the method or device used, such detection is aimed at determining where the cyclic peptide is concentrated in a subject, with such concentration being an indicator of the location of a tumor, organ, or tissue in the subject.

In yet another embodiment, X₃ is an aromatic amino acid such as Tyr, Phe, Trp, or an analog thereof. Suitable Tyr, Phe, and Trp analogs are described above. In still yet another embodiment, the receptor-binding motif is any of the sequence motifs or domains described above. Preferably, the receptor-binding motif is an integrin-binding motif.

In a preferred embodiment, X₃ and Lys in the cyclic peptides described herein have the same configuration. Other amino acid configurations that are within the scope of the present invention are described above.

In another preferred embodiment, X₂ is an integrin-binding motif; X₃ is Tyr, Tyr(Me), or Phe; the ε-amino group of Lys has a benzoyl group conjugated thereto; and X₃ and Lys have an L-configuration. Preferably, the integrin-binding motif has the amino acid sequence Arg-Gly-Asp (RGD) or Asp-Leu-X-X-Leu (DLXXL), where X is any amino acid. Preferably, the benzoyl group is labeled with a radionuclide. Suitable radionuclides for use in labeling the benzoyl group include, without limitation, ¹¹C, ¹³N, ¹⁵O, ¹⁸F, ⁶¹Cu, ⁶²Cu, ⁶⁴Cu, ⁶⁸Ga, 124I, and 131I.

In a particularly preferred embodiment, the cyclic peptide has the following formula: 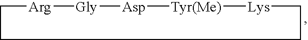
wherein the ε-amino group of Lys has a 4-[¹⁸F]-fluorobenzoyl group conjugated thereto. As such, the cyclic peptide has the amino acid sequence 4-[¹⁸F]-fluorobenzoyl cyclic (RGDY(OMe)K). Such radiolabeled cyclic peptides can be used in, e.g., imaging a tumor, organ, or tissue. In a preferred embodiment, the cyclic peptide adopts a single conformation. In another preferred embodiment, the pi-pi stacking interaction between the fluorobenzoyl group and the aromatic Tyr side-chain restricts (i.e., locks) the cyclic peptide in a single conformation, thereby increasing its affinity and selectively for the αᵥβ₃ integrin receptor.

In addition to their use as imaging agents for imaging tumors, organs, and tissues, the cyclic peptides of the present invention are also suitable for use as therapeutic agents for the treatment of cancer, inflammatory diseases, and autoimmune diseases.

As such, in yet another aspect, the present invention provides a method for treating cancer in a subject in need thereof, the method comprising:
administering to the subject a therapeutically effective amount of a cyclic peptide having the formula: 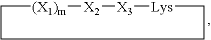
   wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration.

In one embodiment, m is 0 or 1. In another embodiment, m is 2, 3, 4, 5, 6, 7, 8, 9, or 10. In some embodiments, X₁ comprises m amino acids that are independently selected from the group consisting of naturally-occurring amino acids or stereoisomers thereof, unnatural amino acids, and combinations thereof.

In another embodiment, the pi-pi stacking moiety is a benzoyl group, a benzyl group, a naphthoyl group, and a naphthyl group. In certain instances, the pi-pi stacking moiety is labeled with a nuclide, a radionuclide, or a chelating agent. For example, the cyclic peptide can have conjugated thereto a radiolabeled pi-pi stacking moiety such as a 4-[¹⁸F]-fluorobenzoyl group, and the resulting radiolabeled cyclic peptide can be used in radiotherapy, e.g., for treating cancer. Alternatively, the cyclic peptide can have conjugated thereto a labeled pi-pi stacking moiety such as a 4-[¹⁹F]-fluorobenzoyl group, and the resulting labeled cyclic peptide can be used in treating cancer.

Generally, the nuclide or radionuclide can be attached directly to the pi-pi stacking moiety, or alternatively, the nuclide or radionuclide can be bound to a chelating agent attached to the pi-pi stacking moiety. Suitable nuclides for direct conjugation in treating cancer include, without limitation, ¹⁹F. Suitable radionuclides for direct conjugation in treating cancer include, without limitation, ¹⁸F, ¹²⁴I, ¹²⁵i, and ¹³¹I. Suitable radionuclides for use with a chelating agent in treating cancer include, without limitation, ⁴⁷Sc, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, e.g., the chelating agents described above.

In yet another embodiment, X₃ is an aromatic amino acid such as Tyr, Phe, Trp, or an analog thereof. Suitable Tyr, Phe, and Trp analogs are described above. In still yet another embodiment, the receptor-binding motif is any of the sequence motifs or domains described above. Preferably, the receptor-binding motif is an integrin-binding motif.

In a preferred embodiment, X₃ and Lys in the cyclic peptides described herein have the same configuration. Other amino acid configurations that are within the scope of the present invention are described above.

Types of cancers that are suitable for treatment using the cyclic peptides of the present invention are described above. Preferably, the cyclic peptides are used for treating cutaneous melanoma, glioblastoma, or Kaposi's sarcoma. The cyclic peptides are also particularly useful for treating breast, oral, or prostate cancer.

In a preferred embodiment, X₂ is an integrin-binding motif; X₃ is Tyr, Tyr(Me), or Phe; the ε-amino group of Lys has a benzoyl group conjugated thereto; and X₃ and Lys have an L-configuration. Preferably, the integrin-binding motif has the amino acid sequence Arg-Gly-Asp (RGD) or Asp-Leu-X-X-Leu (DLXXL), where X is any amino acid. In certain instances, the benzoyl group is labeled with a nuclide. Suitable nuclides for use in labeling the benzoyl group include, without limitation, ¹⁹F. In certain other instances, the benzoyl group is labeled with a radionuclide. Suitable radionuclides for use in labeling the benzoyl group include, without limitation, ¹⁸F, ⁶⁷Cu, and ¹³¹I.

In a particularly preferred embodiment, the cyclic peptide has the following formula: 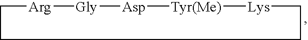
wherein the ε-amino group of Lys has a 4-[¹⁸F]-fluorobenzoyl group or a 4-[¹⁹F]-fluorobenzoyl group conjugated thereto. In certain instances, the cyclic peptide has the amino acid sequence 4-[¹⁹F]-fluorobenzoyl cyclic (RGDY(OMe)K). In certain other instances, the cyclic peptide has the amino acid sequence 4-[¹⁸F]-fluorobenzoyl cyclic (RGDY(OMe)K). These cyclic peptides can be used in treating any of the above-described cancers, e.g., cutaneous melanoma, glioblastoma, or Kaposi's sarcoma. In a preferred embodiment, the cyclic peptide adopts a single conformation. In another preferred embodiment, the pi-pi stacking interaction between the fluorobenzoyl group and the aromatic Tyr side-chain restricts (i.e., locks) the cyclic peptide in a single conformation, thereby increasing its affinity and selectively for the αᵥβ₃ integrin receptor.

In still yet another aspect, the present invention provides a method for treating an inflammatory or autoimmune disease in a subject in need thereof, the method comprising:
administering to the subject a therapeutically effective amount of a cyclic peptide having the formula: 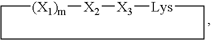
   wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration.

In one embodiment, m is 0 or 1. In another embodiment, m is 2, 3, 4, 5, 6, 7, 8, 9, or 10. In some embodiments, X₁ comprises m amino acids that are independently selected from the group consisting of naturally-occurring amino acids or stereoisomers thereof, unnatural amino acids, and combinations thereof.

In another embodiment, the pi-pi stacking moiety is selected from the group consisting of a benzoyl group, a benzyl group, a naphthoyl group, and a naphthyl group. In certain instances, the pi-pi stacking moiety is labeled with a nuclide, a radionuclide, or a chelating agent. For example, the cyclic peptide can have conjugated thereto a radiolabeled pi-pi stacking moiety such as a 4-[¹⁸F]-fluorobenzoyl group, and the resulting radiolabeled cyclic peptide can be used in radiotherapy, e.g., for treating an inflammatory or autoimmune disease. Alternatively, the cyclic peptide can have conjugated thereto a labeled pi-pi stacking moiety such as a 4-[¹⁹F]-fluorobenzoyl group, and the resulting labeled cyclic peptide can be used in treating an inflammatory or autoimmune disease.

Generally, the nuclide or radionuclide can be attached directly to the pi-pi stacking moiety, or alternatively, the nuclide or radionuclide can be bound to a chelating agent attached to the pi-pi stacking moiety. Suitable nuclides for direct conjugation in treating an inflammatory or autoimmune disease include, without limitation, ¹⁹F. Suitable radionuclides for direct conjugation in treating an inflammatory or autoimmune disease include, without limitation, ¹⁸F, ¹²⁴I, ¹²⁵I, and ¹³¹I. Suitable radionuclides for use with a chelating agent in treating an inflammatory or autoimmune disease include, without limitation, ⁴⁷Sc, ⁶⁷Cu, ⁸⁹Sr, ⁸⁶Y, ⁸⁷Y,⁹⁰Y, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, e.g., the chelating agents described above.

In yet another embodiment, X₃ is an aromatic amino acid such as Tyr, Phe, Trp, or an analog thereof. Suitable Tyr, Phe, and Trp analogs are described above. In still yet another embodiment, the receptor-binding motif is any of the sequence motifs or domains described above. Preferably, the receptor-binding motif is an integrin-binding motif.

In a preferred embodiment, X₃ and Lys in the cyclic peptides described herein have the same configuration. Other amino acid configurations that are within the scope of the present invention are described above.

Types of inflammatory or autoimmune diseases that are suitable for treatment using the cyclic peptides of the present invention are described above. Preferably, the cyclic peptides are used for treating rheumatoid arthritis.

In a preferred embodiment, X₂ is an integrin-binding motif; X₃ is Tyr, Tyr(Me), or Phe; the ε-amino group of Lys has a benzoyl group conjugated thereto; and X₃ and Lys have an L-configuration. Preferably, the integrin-binding motif has the amino acid sequence Arg-Gly-Asp (RGD) or Asp-Leu-X-X-Leu (DLXXL), where X is any amino acid. In certain instances, the benzoyl group is labeled with a nuclide. Suitable nuclides for use in labeling the benzoyl group include, without limitation, ¹⁹F. In certain other instances, the benzoyl group is labeled with a radionuclide. Suitable radionuclides for use in labeling the benzoyl group include, without limitation, ¹⁸F, ⁶⁷Cu, and ¹³¹I.

In a particularly preferred embodiment, the cyclic peptide has the following formula: 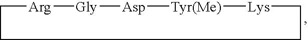
wherein the ε-amino group of Lys has a 4-[¹⁸F]-fluorobenzoyl group or a 4-[¹⁹F]-fluorobenzoyl group conjugated thereto. In certain instances, the cyclic peptide has the amino acid sequence 4-[¹⁹F]-fluorobenzoyl cyclic (RGDY(OMe)K). In certain other instances, the cyclic peptide has the amino acid sequence 4-[18F]-fluorobenzoyl cyclic (RGDY(OMe)K). These cyclic peptides can be used in treating any of the above-described inflammatory or autoimmune disease, e.g., rheumatoid arthritis. In a preferred embodiment, the cyclic peptide adopts a single conformation. In another preferred embodiment, the pi-pi stacking interaction between the fluorobenzoyl group and the aromatic Tyr side-chain restricts (i.e., locks) the cyclic peptide in a single conformation, thereby increasing its affinity and selectively for the αᵥβ₃ integrin receptor.

In a further aspect, the present invention provides a method for identifying a receptor-binding cyclic peptide, the method comprising:
(a) contacting a receptor or fragment thereof with a cyclic peptide having the formula: 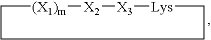
   wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration; and
(b) determining the binding of the cyclic peptide to the receptor or fragment thereof

In one embodiment, m is 0 or 1. In another embodiment, m is 2, 3, 4, 5, 6, 7, 8, 9, or 10. In another embodiment, X₁ comprises m amino acids that are independently selected from the group consisting of naturally-occurring amino acids or stereoisomers thereof, unnatural amino acids, and combinations thereof. In yet another embodiment, the pi-pi stacking moiety is a benzoyl group, a benzyl group, a naphthoyl group, or a naphthyl group. In certain instances, the pi-pi stacking moiety is labeled with a nuclide, a radionuclide, or a chelating agent as described above.

In another embodiment, X₃ is an aromatic amino acid such as Tyr, Phe, Trp, or an analog thereof. Suitable Tyr, Phe, and Trp analogs are described above. In yet another embodiment, the receptor-binding motif is any of the sequence motifs or domains described above; Preferably, the receptor-binding motif is an integrin-binding motif.

In a further embodiment, X₃ and Lys in the cyclic peptides described herein have the same configuration. Other amino acid configurations that are within the scope of the present invention are described above.

In a preferred embodiment, the cyclic peptide adopts a single conformation. In another preferred embodiment, the pi-pi stacking interaction between the pi-pi stacking moiety and the aromatic amino acid restricts (i.e., locks) the cyclic peptide in a single conformation, thereby increasing its affinity and selectively for the receptor or fragment thereof.

Suitable receptors for use in the present invention include, without limitation, an integrin receptor, a growth factor receptor (e.g., epidermal growth factor receptor), a cytokine receptor (e.g., an interleukin receptor), a TGF receptor (e.g., TGF-β receptor), a tumor necrosis factor receptor (e.g., TNF-α receptor), a G-protein coupled receptor (e.g., neurotransmitter receptors, chemokine receptors, olfactory receptors, etc.), a scavenger receptor (e.g., CD36), a lipoprotein receptor (e.g., LDL receptor), other immune cell receptors (e.g., T cell receptor), combinations thereof, and fragments thereof.

Suitable assays for identifying the receptor-binding cyclic peptide include, without limitation, an enzyme-linked immunosorbent assay (ELISA) or an adhesion assay, e.g., as described in Example 2 below; an assay for detecting labeled or radiolabeled peptides; an assay for detecting fluorescent peptides; a chemiluminescence assay; high pressure liquid chromatography (HPLC); nuclear magnetic resonance (NMR) spectroscopy; and mass spectrometry (e.g., MALDI/MS, MALDI-TOF/MS, tandem MS, etc.). One skilled in the art will appreciate suitable conditions for performing the assays, e.g., suitable binding, washing, and/or detecting conditions, etc. In some embodiments, a plurality of cyclic peptides are individually tested for binding to the receptor of interest, e.g., in the wells of a microtiter plate, in which the receptor or fragment thereof is contacted with a different cyclic peptide in each well. Alternatively, a plurality of cyclic peptides are individually tested for binding to the receptor of interest using array-based technology. In other embodiments, the receptor or fragment thereof is contacted with a plurality of cyclic peptides, and any binding between one or more of the plurality of cyclic peptides and the receptor or fragment thereof is determined.

In certain embodiments, the above-described method for identifying a receptor-binding cyclic peptide further comprises repeating steps (a) and (b). As a non-limiting example, the receptor or fragment thereof can be contacted with a series of cyclic peptides until a cyclic peptide with the desired receptor-binding affinity and/or selectivity is identified. As such, one skilled in the art will appreciate that a plurality of cyclic peptides can be screened using such an iterative approach to facilitate the discovery of those cyclic peptides with greater affinity and/or selectivity for the receptor of interest.

In additional aspects, the present invention provides a kit for imaging a tumor, organ, or tissue in a subject, for treating cancer in a subject in need thereof, or for treating an inflammatory or autoimmune disease in a subject in need thereof, the kit comprising:
(a) a container holding a cyclic peptide having the formula: 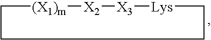
   wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration; and
(b) directions for use of the cyclic peptide in imaging a tumor, organ, or tissue, in treating cancer, or in treating an inflammatory or autoimmune disease.

In one embodiment, m is 0 or 1. In another embodiment, m is 2, 3, 4, 5, 6, 7, 8, 9, or 10. In some embodiments, X₁ comprises m amino acids that are independently selected from the group consisting of naturally-occurring amino acids or stereoisomers thereof, unnatural amino acids, and combinations thereof.

In another embodiment, the pi-pi stacking moiety is selected from the group consisting of a benzoyl group, a benzyl group, a naphthoyl group, and a naphthyl group. When the kit is used for imaging a tumor, organ, or tissue in a subject, the pi-pi stacking moiety is typically labeled with an imaging moiety such as a nuclide, a radionuclide, a chelating agent, a fluorophore, an antibody, and biotin or a derivative thereof. Preferably, the imaging moiety is a radionuclide and the radiolabeled cyclic peptide is used for radioimaging. For example, the cyclic peptide can have conjugated thereto a radiolabeled pi-pi stacking moiety such as a 4-[¹⁸F]-fluorobenzoyl group, and the resulting radiolabeled cyclic peptide can be used in imaging a tumor, organ, or tissue using any radioimaging technique known in the art. When the kit is used for treating cancer or for treating an inflammatory or autoimmune disease, the pi-pi stacking moiety is typically labeled with a nuclide, a radionuclide, or a chelating agent. Preferably, the imaging moiety is a radionuclide and the radiolabeled cyclic peptide is used for radiotherapy. For example, the cyclic peptide can have conjugated thereto a radiolabeled pi-pi stacking moiety such as a 4-[¹⁸F]-fluorobenzoyl group, and the resulting radiolabeled cyclic peptide can be used in treating cancer or in treating an inflammatory or autoimmune disease. Alternatively, the cyclic peptide can have conjugated thereto a labeled pi-pi stacking moiety such as a 4-[¹⁹F]-fluorobenzoyl group, and the resulting labeled cyclic peptide can be used in treating cancer or in treating an inflammatory or autoimmune disease.

Generally, the nuclide or radionuclide can be attached directly to the pi-pi stacking moiety, or alternatively, the nuclide or radionuclide can be bound to a chelating agent attached to the pi-pi stacking moiety. Suitable radionuclides for direct conjugation in imaging a tumor, organ, or tissue include, without limitation, ¹¹C, ¹³N, ¹⁵O, ¹⁸F, ¹²⁴I, and ¹³¹I. Suitable radionuclides for use with a chelating agent in imaging a tumor, organ, or tissue include, without limitation, ⁵⁵Co, ⁶⁰Cu, 61Cu, ⁶²Cu, ⁶⁴Cu, ⁶⁶Ga, ⁶⁷Ga, ⁶⁸Ga, ⁸²Rb, ⁸⁶Y, ⁸⁷Y, 90Y, ¹¹¹In, ^{99m}Tc, ²⁰¹Tl, and mixtures thereof. Suitable nuclides for direct conjugation in treating cancer or in treating an inflammatory or autoimmune disease include, without limitation, ¹⁹F. Suitable radionuclides for direct conjugation in treating cancer or in treating an inflammatory or autoimmune disease include, without limitation, ¹⁸F, ¹²⁴I, ¹²⁵I, and ¹³¹I. Suitable radionuclides for use with a chelating agent in treating cancer or in treating an inflammatory or autoimmune disease include, without limitation, ⁴⁷Sc, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi and mixtures thereof. Suitable chelating agents include, e.g., the chelating agents described above.

In yet another embodiment, X₃ is an aromatic amino acid such as Tyr, Phe, Trp, or an analog thereof. Suitable Tyr, Phe, and Trp analogs are described above. In yet another embodiment, the receptor-binding motif is any of the sequence motifs or domains described above. Preferably, the receptor-binding motif is an integrin-binding motif.

In a preferred embodiment, X₃ and Lys in the cyclic peptides described herein have the same configuration. Other amino acid configurations that are within the scope of the present invention are described above.

The container holding the cyclic peptide in the kits described herein can be any container suitable for holding one or more unit dosage forms of the cyclic peptides of the present invention. For example, when the cyclic peptide is in the form of a powder, solution, suspension, or emulsion, the container can be, e.g., a vial, an ampoule, or a syringe. Alternatively, when the cyclic peptide is in the form of a tablet, pill, capsule, lozenge, pellet, candy, or gum, any container known in the art for packaging such unit dosage forms can be used. Further, when the cyclic peptide is in the form of a cream, ointment, lotion, gel, spray, or foam, the container can be, e.g., a tube, a bottle, or an aerosol can. Directions for the use of the cyclic peptides of the present invention in imaging a tumor, organ, or tissue, in treating cancer, or in treating an inflammatory or autoimmune disease are also supplied with the kits described herein. In certain instances, the directions are intended for a clinician such as a general practitioner or a specialist involved with imaging or treating the subject. In certain other instances, the directions are intended for the subject.

IV. Integrin Expression in Disease

High-affinity receptors are frequently over-expressed in many diseases, making them important targets for both diagnosis and therapy. One such family of receptors are the integrins. Integrins are a family of heterodimeric molecules expressed on the surface of eukaryotic cells and serve as receptors for glycoproteins in the extracellular matrix (ECM) or other cell surface proteins. Integrins translate the binding of ECM ligands into intracellular messages that allow cells to adhere to, spread on, and migrate through the stroma (Webb et al., Methods Cell Biol., 69:341-358 (2002)). As a result, integrins are essential for both normal and pathological processes including cell growth, differentiation, migration, tumorigenesis, and metastasis. Table 1 below lists several integrins and the diseases associated with them.

TABLE 1
Diseases associated with integrin upregulation.

| Integrin | Disease |
|---|---|
| αᵥβ₃ | Ovarian carcinoma |
| | Breast cancer |
| | Bone metastasis in prostate cancer |
| | Melanoma |
| | Glioblastoma |
| | Kaposi's sarcoma |
| | Rheumatoid arthritis |
| | Cardiovascular disease |
| αᵥβ₃ | Kidney disease |
| | Oral squamous cell carcinoma |
| | Ovarian cancer |
| | Colon cancer |
| α₂β₁,α₃β₁ | Kidney disease, e.g., diabetic |
| | glomerulosclerosis |
| α_{IIb}β₃ | Deep vein thrombosis |
| | Myocardial infarction |
| α₄β₁,α₄β₇ | Multiple sclerosis |
| | Rheumatoid arthritis |
| | Inflammatory bowel disease |

The present invention advantageously allows for the early detection and treatment of many diseases by providing cyclic peptides with improved affinity and selectivity (e.g., by at least a factor of 100) for specific integrin receptors. In particular, the cyclic peptides of the present invention that bind to αᵥβ₃ integrin are well-suited for use as in vivo molecular imaging probes to detect diseases associated with this integrin at an earlier stage. For example, αᵥβ₃ integrin has been shown to promote cell growth, inhibit apoptosis, increase protease production, promote invasion of certain cancers, and play an essential role in angiogenesis (Brooks et al., Cell, 79:1157-1164 (1994)). In fact, αᵥβ₃ integrin is not expressed strongly on resting tissues but is significantly increased on several tumor types including, but not limited to, cutaneous melanoma (Albelda et al., Faseb J., 4:2868-2880 (1990)), glioblastoma (Gladson et al., J. Clin. Invest., 88:1924-1932 (1991)), and Kaposi's sarcoma (Ensoli et al., Eur. J. Cancer, 37:1251-1269 (2001)). Inhibition of αᵥβ₃ integrin can block subcutaneous growth of melanoma xenografts. In addition, αᵥβ₃ integrin is expressed de novo on all solid cancers.

The extracellular globular domain of integrins associate with their ligands via short peptide motifs. The first of these ligand-recognition sites to be identified was the RGD motif from the smallest active fragment of fibronectin (Pierschbacher et al., Nature, 309:30-33 (1984)). The RGD motif has been identified in many extracellular matrix and serum proteins including, but not limited to, fibronectin, vitronectin, laminin, fibrogen, von Willebrand factor, and certain collagens. The principal integrins that bind via the RGD motif include αᵥβ₁, αᵥβ₃, αᵥβ₅, αᵥβ₆, α_{IIb}β₃, α₅β₁, and αᵥβ₁. As a result, the structural locking mechanism of the present invention can be used to generate cyclic peptides that bind to specific RGD-binding integrins with significantly improved localizing and/or targeting potential. For example, the cyclic peptides of the present invention that interact with αᵥβ₃ integrin can be used to detect αᵥβ₃ that is expressed de novo on angiogenic blood vessels of tumors or regions of tissue repair. Thus, the cyclic peptides of the present invention can be used as in vivo molecular imaging probes and/or therapeutic agents to identify and/or treat cancer (e.g., occult metastases), inflammatory diseases (e.g., rheumatoid arthritis), autoimmune diseases, cardiovascular diseases (e.g., restenosis, coronary heart disease, myocardial infarction, stroke, cardiomyopathy, pericarditis, high blood pressure, and the like), and kidney diseases (e.g., diabetic glomeruloscierosis, nephritis, nephropathy, cystic kidney disease, and the like). The broad and beneficial applications of these cyclic peptides can have a positive impact on patient management for any of the above-mentioned diseases.

The αᵥβ₆ integrin, which is a receptor for fibronectin, tenascin, vitronectin, and the latency associated peptide (LAP) of TGF-β, is expressed at very low or undetectable levels in only a subset of epithelial cells in normal adult tissues (Breuss et al., J. Cell Sci., 108:2241 -2251 (1995)). However, αᵥβ₆ integrin expression is increased dramatically during development, following injury or inflammation, or in a variety of epithelial neoplasms. For example, keratinocytes show de novo expression of αᵥβ₆ integrin in both oral and skin wounds (Breuss et al., supra; Clark et al., Am. J. Path., 148:1407-1421 (1996)). In addition, αᵥβ₆ integrin plays an active role in tumor invasion because its expression is often higher at the invasive margins of oral squamous cell carcinomas. As a result, αᵥβ₆ integrin is an excellent target for both imaging and therapy of diseases such as oral cancer, ovarian cancer, and colon cancer using the cyclic peptides of the present invention. As such, the structural locking mechanism of the present invention can be used to generate cyclic peptides containing the DLXXL motif that bind to αᵥβ₆ integrin with improved affinity and selectivity, thereby providing significantly better localizing and/or targeting potential.

V. Methods of Administration

The cyclic peptides of the present invention have particular utility in human and veterinary imaging, therapeutic, and diagnostic applications. For example, the cyclic peptides can be used for imaging tumors, organs, or tissues and for treating cancer, inflammatory diseases, autoimmune diseases, or cardiovascular diseases.

Administration of the cyclic peptides of the present invention with a suitable pharmaceutical excipient as necessary can be carried out via any of the accepted modes of administration. Thus, administration can be, for example, intravenous, topical, subcutaneous, transcutaneous, transdermal, transmucosal, intramuscular, oral, intra-joint, parenteral, intra-arteriole, intradermal, intraventricular, intracranial, intraperitoneal, intratracheal, intralesional, intranasal, or by inhalation. Moreover, where injection is to treat a tumor, administration may be directly to the tumor and/or into tissues surrounding the tumor.

The compositions containing a cyclic peptide or a combination of cyclic peptides of the present invention may be administered repeatedly, e.g., at least 2, 3, 4, 5, 6, 7, 8, or more times, or the composition may be administered by continuous infusion. Suitable sites of administration include, but are not limited to, skin, bronchial, gastrointestinal, oral, anal, vaginal, eye, and ear. The formulations may take the form of solid, semi-solid, lyophilized powder, or liquid dosage forms, such as, for example, tablets, pills, capsules, lozenges, pellets, candies, gums, powders, solutions, suspensions, emulsions, suppositories, retention enemas, creams, ointments, lotions, gels, aerosols, or the like, preferably in unit dosage forms suitable for simple administration of precise dosages.

The term “unit dosage form” refers to physically discrete units suitable as unitary dosages for human subjects and other mammals (e.g., dogs, cats, livestock, etc.), each unit containing a predetermined quantity of active material calculated to produce the desired onset, tolerability, and/or therapeutic effects, in association with one or more suitable pharmaceutical excipients or carriers. Methods for preparing such dosage forms are known or will be apparent to those skilled in the art. For example, in some embodiments, a chewing gum dosage form of the present invention can be prepared according to the procedures set forth in U.S. Pat. No. 4,405,647. In other embodiments, a tablet, lozenge, or candy dosage form of the present invention can be prepared according to the procedures set forth, for example, in Remington: The Science and Practice of Pharmacy, 20^{th} Ed., Lippincott, Williams & Wilkins (2003); Pharmaceutical Dosage Forms, Volume 1: Tablets, 2^{nd} Ed., Marcel Dekker, Inc., New York, N.Y. (1989); and similar publications. The dosage form to be administered will, in any event, contain a quantity of the cyclic peptide or combination of cyclic peptides in a therapeutically effective amount for imaging a tumor, organ, or tissue or for relief of a condition being treated (e.g., cancer, inflammatory disease, autoimmune disease, cardiovascular disease, etc.) when administered in accordance with the teachings of the present invention. In addition, pharmaceutically acceptable salts of the cyclic peptides of the present invention may be prepared and included in the compositions using standard procedures known to those skilled in the art of synthetic organic chemistry and described, e.g., by J. March, Advanced Organic Chemistry: Reactions, Mechanisms and Structure, 4^{th} Ed. (New York: Wiley-Interscience, 1992). More concentrated compositions may also be prepared, from which the more dilute unit dosage compositions may then be produced. The more concentrated compositions thus will contain substantially more than, e.g., at least 1, 2, 3, 4, 5, 6, 7, 8, 9, 10, or more times the amount of a cyclic peptide or a combination of cyclic peptides.

The compositions typically include a conventional pharmaceutical carrier or excipient and may additionally include other medicinal agents, carriers, adjuvants, diluents, tissue permeation enhancers, solubilizers, sweetening agents, flavoring agents, protecting agents, plasticizers, waxes, elastomeric solvents, filler materials, preservatives, lubricating agents, wetting agents, emulsifying agents, suspending agents, coloring agents, disintegrating agents, and the like. The compositions may also comprise biodegradable polymer beads, dextran, and cyclodextrin inclusion complexes. Preferably, the composition contains from about 0.001% to about 90%, preferably from about 0.01% to about 75%, more preferably from about 0.1% to 50%, and still more preferably from about 0.1% to 10% by weight of a cyclic peptide of the present invention or a combination thereof, with the remainder consisting of suitable pharmaceutical carriers, excipients, and/or other ingredients. Appropriate excipients can be tailored to the particular composition and route of administration by methods well known in the art, e.g., Remington: The Science and Practice of pharmacy, supra.

Examples of suitable carriers or excipients include, without limitation, lactose, dextrose, sucrose, glucose, powdered sugar, sorbitol, mannitol, xylitol, starches, acacia gum, xanthan gum, guar gum, tara gum, mesquite gum, fenugreek gum, locust bean gum, ghatti gum, tragacanth gum, inositol, molasses, maltodextrin, extract of Irish moss, panwar gum, mucilage of isapol husks, Veegum®, larch arabogalactan, calcium silicate, calcium phosphate, dicalcium phosphate, calcium sulfate, kaolin, sodium chloride, polyethylene glycol, alginates, gelatin, microcrystalline cellulose, polyvinylpyrrolidone, cellulose, water, saline, syrup, methylcellulose, ethylcellulose, hydroxypropylnethylcellulose, carboxymethylcellulose, polyacrylic acids such as Carbopols, e.g., Carbopol 941, Carbopol 980, Carbopol 981, and gum bases such as Pharmagum™ M, S, or C (SPI Pharma Group; New Castle, Del.), etc. Typically, the compositions of the present invention comprise from about 10% to about 90% by weight of the carrier, the excipient, or combinations thereof.

Examples of suitable lubricating agents include, without limitation, magnesium stearate, calcium stearate, zinc stearate, stearic acid, simethicone, silicon dioxide, talc, hydrogenated vegetable oil, polyethylene glycol, mineral oil, and combinations thereof. Typically, the compositions of the present invention comprise from about 0% to about 10% by weight of the lubricating agent.

Examples of suitable preservatives include, without limitation, methyl-, ethyl-, and propyl-hydroxy-benzoates, butylated hydroxytoluene, and butylated hydroxyanisole. Typically, the compositions of the present invention comprise from about 0% to about 10% by weight of the preservative.

Sweetening agents can be used to improve the palatability of the composition by masking any unpleasant tastes it may have. Examples of suitable sweetening agents include, without limitation, compounds selected from the saccharide family such as the mono-, di-, tri-, poly-, and oligosaccharides; sugars such as sucrose, glucose (corn syrup), dextrose, invert sugar, fructose, maltodextrin, and polydextrose; saccharin and salts thereof such as sodium and calcium salts; cyclamic acid and salts thereof; dipeptide sweeteners; chlorinated sugar derivatives such as sucralose and dihydrochalcone; sugar alcohols such as sorbitol, sorbitol syrup, mannitol, xylitol, hexa-resorcinol, and the like, and combinations thereof. Hydrogenated starch hydrolysate, and the potassium, calcium, and sodium salts of 3,6-dihydro-6-methyl-1-1,2,3-oxathiazin-4-one-2,2-dioxide may also be used. Typically, the compositions of the present invention comprise from about 0% to about 80% by weight of the sweetening agent.

Flavoring agents can also be used to improve the palatability of the composition. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g., white, milk, dark), vanilla, caramel, coffee, hazelnut, combinations thereof, and the like. Typically, the compositions of the present invention comprise from about 0% to about 10% by weight of the flavoring agent.

Coloring agents can be used to color code the composition, for example, to indicate the type and dosage of the cyclic peptide or combination of cyclic peptides contained therein. Suitable coloring agents include, without limitation, natural and/or artificial compounds such as FD & C coloring agents, natural juice concentrates, pigments such as titanium oxide, silicon dioxide, and zinc oxide, combinations thereof, and the like. Typically, the compositions of the present invention comprise from about 0% to about 10% by weight of the coloring agent.

Non-limiting examples of plasticizers suitable for use in the present invention include lecithin, mono- and diglycerides, lanolin, stearic acid, sodium stearate, potassium stearate, glycerol triacetate, glycerol monostearate, glycerin, and combinations thereof. Typically, the compositions of the present invention comprise from about 0% to about 20% by weight of the plasticizer.

Examples of suitable elastomeric solvents include, without limitation, rosins and resins such as methyl, glycerol, and pentaerythritol esters of rosins, modified rosins such as hydrogenated, dimerized or polymerized rosins, or combinations thereof (e.g., pentaerythritol ester of partially hydrogenated wood rosin, pentaerythritol ester of wood rosin, glycerol ester of wood rosin, glycerol ester of partially dimerized rosin, glycerol ester of polymerized rosin, glycerol ester of tall oil rosin, glycerol ester of wood rosin and partially hydrogenated wood rosin and partially hydrogenated methyl ester of rosin such as polymers of alpha-pinene or beta-pinene, terpene resins including polyterpene, and combinations thereof). Typically, the compositions of the present invention comprise from about 0% to about 25% by weight of the elastomeric solvent.

Examples of suitable filler materials include, without limitation, calcium carbonate, magnesium silicate (i.e., talc), dicalcium phosphate, metallic mineral salts (e.g., alumina, aluminum hydroxide, and aluminum silicates), and combinations thereof. Typically, the compositions of the present invention comprise from about 0% to about 20% by weight of the filler material.

Examples of suitable waxes include, without limitation, beeswax and microcrystalline wax, fats or oils such as soybean and cottonseed oil, and combinations thereof. Typically, the compositions of the present invention comprise from about 0% to about 20% by weight of the wax.

Examples of suitable protecting agents include, without limitation, calcium stearate, glycerin monostearate, glyceryl behenate, glyceryl palmitostearate, hydrogenated castor oil, hydrogenated vegetable oil type I, light mineral oil, magnesium lauryl sulfate, magnesium stearate, mineral oil, poloxamer, polyethylene gycol, sodium benzoate, sodium chloride, sodium lauryl sulfate, stearic acid, cab-o-sil, talc, zinc stearate, and combinations thereof. Typically, the compositions of the present invention comprise from about 0% to about 50% by weight of the protecting agent.

Examples of suitable disintegrating agents include, without limitation, crospovidone, croscarmellose sodium, other cross-linked cellulose polymers, and combinations thereof. Typically, the compositions of the present invention comprise from about 0% to about 20% by weight of the disintegrating agent.

Liquid compositions can be prepared by dissolving or dispersing the cyclic peptide or combination of cyclic peptides and optionally one or more pharmaceutically acceptable adjuvants in a carrier such as, for example, aqueous saline (e.g., 0.9% w/v sodium chloride), aqueous dextrose, glycerol, ethanol, and the like, to form a solution, suspension, or emulsion, e.g., for oral, topical, or intravenous administration.

For oral administration, the compositions can be in the form of tablets, pills, capsules, lozenges, candies, emulsions, suspensions, solutions, syrups, sprays, powders, quick-dissolving formulations, and sustained-release formulations. Suitable carriers or excipients for oral administration include, e.g., pharmaceutical grades of mannitol, lactose, starch, magnesium stearate, sodium saccharine, talcum, cellulose, glucose, gelatin, sucrose, magnesium carbonate, and the like.

For rectal administration, the compositions can be in the form of a suppository disposed, for example, in a polyethylene glycol (PEG) carrier. The cyclic peptides of the present invention can also be formulated into a retention enema.

For topical administration, the compositions of the present invention can be in the form of lotions, gels, creams, aerosols, jellies, solutions, suspensions, emulsions, ointments, and transdermal patches. For delivery by inhalation, the composition can be delivered as a dry powder or in liquid form via a nebulizer. For parenteral administration, the compositions can be in the form of sterile injectable solutions and sterile packaged powders. Preferably, injectable solutions are formulated at a pH of about 4.5 to about 7.5.

The compositions of the present invention can also be provided in a lyophilized form. Such compositions may include a buffer, e.g., bicarbonate, for reconstitution prior to administration, or the buffer may be included in the lyophilized composition for reconstitution with, e.g., water. The lyophilized composition may further comprise a suitable vasoconstrictor, e.g., epinephrine. The lyophilized composition can be provided in a syringe, optionally packaged in combination with the buffer for reconstitution, such that the reconstituted composition can be immediately administered to a patient.

Generally, administered dosages will be effective to deliver picomolar to micromolar concentrations of the cyclic peptide or combination of cyclic peptides to the appropriate site or sites. However, one skilled in the art understands that the dose administered will vary depending on a number of factors, including, but not limited to, the particular cyclic peptide or set of cyclic peptides to be administered, the mode of administration, the type of application (e.g., imaging, therapeutic), the age of the patient, and the physical condition of the patient. Preferably, the smallest dose and concentration required to produce the desired result should be used. Dosage should be appropriately adjusted for children, the elderly, debilitated patients, and patients with cardiac and/or liver disease. Further guidance can be obtained from studies known in the art using experimental animal models for evaluating dosage. However, one skilled in the art understands that the increased receptor affinity and selectively associated with the cyclic peptides of the present invention permits a wider margin of safety for dosage concentrations and for repeated dosing.

### VI. EXAMPLES

The following examples are offered to illustrate, but not to limit, the claimed invention.

### Example 1

Synthesis of RGD Peptides.

This example illustrates the synthesis of the RGD peptides of the present invention.

The following Fmoc amino acids were purchased from Calbiochem-Novabiochem Ltd. (Nottingham, UK): Fmoc-Lys-(Mtt)-OH, Fmoc-Lys-(alloc)-OH, Fmoc-Arg-(Pbf)-OH, Fmoc-Gly-OH, Fmoc-Asp-(OtBu)-OH, Fmoc-Phe-OH, Fmoc-D-Phe-OH, Fmoc-Glu-(allyl)-OH, Fmoc-Tyr(OMe)-OH, and Fmoc-D-Tyr(OtBu)-OH. The acid labile linker resins 5-(4-aminomethyl-3,5-dimethoxyphenoxy)valeryl-PEG/PS (PAL-PEG/PS) and Fmoc-(Asp)-(PEG/PS)-(Oallyl) were purchased from Applied Biosystems (Warrington, UK). 1-hydroxybenzotriazole (HOBt), 1,3-Diisopropylcarbodiimide (DIPCDI), N,N-diisopropylethylamine (DIPEA), and piperidine (PIP) were purchased from Fluka Chemicals (Dorset, UK). Analar grade water, methanol, dichloromethane, and dimethylformamide (DMF) were purchased from BDH (Dorset, UK). HATU was purchased from Applied Biosystems (Warrington, UK). Palladium tetrakis triphenylphospine (Pd(PPh₃)₄), triphenylphosphine (PPh₃), trifluoroaceticacid (TFA), triisopropylsilane (TIPS), and picryl sulfonic acid were purchased from Aldrich Chemical Company (Dorset, UK).

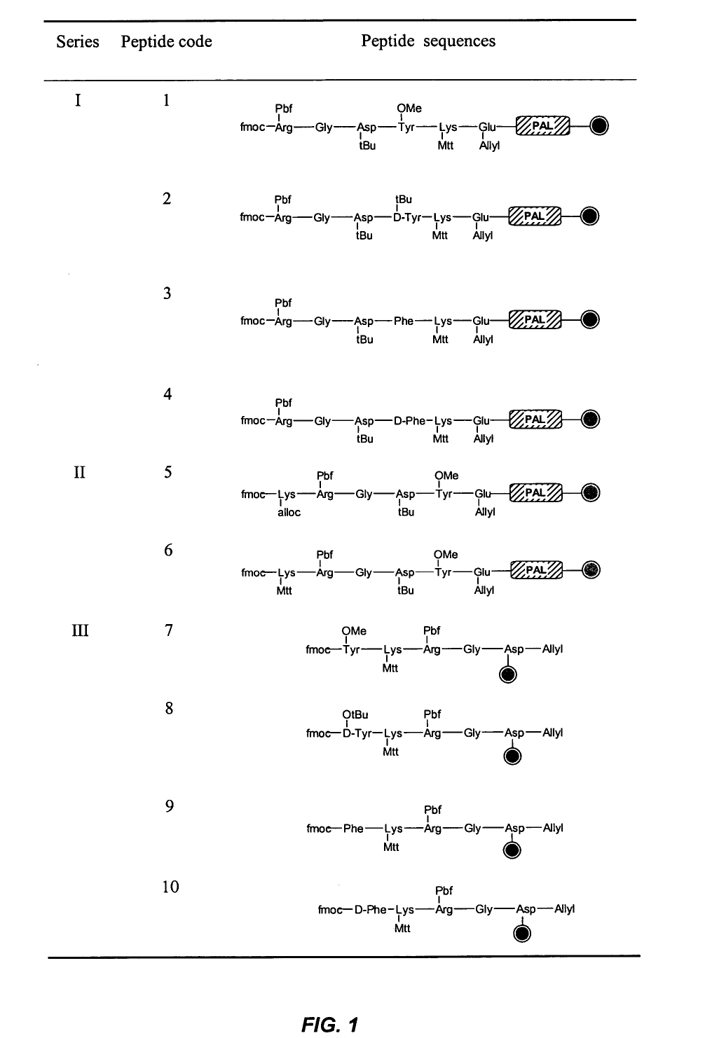

Peptides containing the RGD motif were synthesized using Fmoc chemistry (see, FIG. 1). Three synthetic strategies were used to synthesize a series of hexapeptides (Series I and II in FIG. 1) and pentapeptides (Series III in FIG. 1) containing the RGD motif. Side-chain protection of Glu or Lys residues was afforded by an allyl or alloc protecting group, respectively, and was selectively removed using Pd(PPh₃)₄. Amino-terminal protection was afforded by the base labile Fmoc group.

The peptides in Series I are hexapeptides containing the RGD motif and have the formula Arg-Gly-Asp-X-Lys-Glu. Side-chain protection was afforded using Fmoc-Arg-(Pbf)-OH, Fmoc-Asp-(tBu)-OH, Fmoc-Lys-(Mtt)-OH, and Fmoc-Glu-(Allyl)-OH. The amino acid residue at position X was varied between the L- or D-forms of the hydrophobic Phe and Tyr residues. D-Phe and D-Tyr were used to improve the in vivo stability of the peptide. On-resin cyclization was performed between the amino terminus of the peptide and the ω-carboxyl group of glutamic acid (Glu) in an end to side-chain fashion as shown in Scheme 1 below to yield the cyclic peptide.

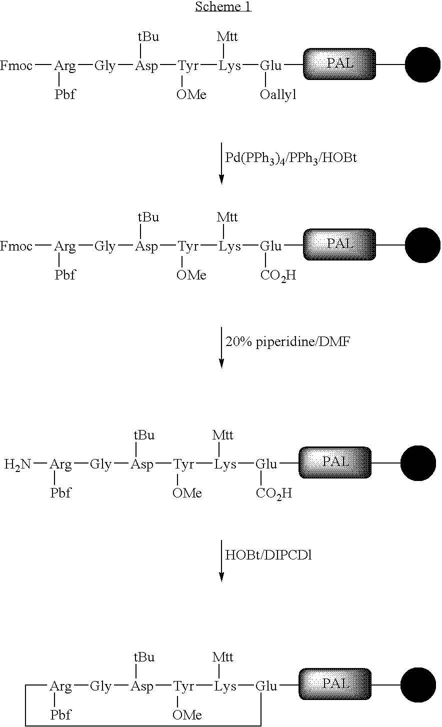

The peptides in Series II are hexapeptides containing the RGD motif and have the formula Lys-Arg-Gly-Asp-Tyr-Glu. Side-chain protection of Arg, Asp, and Glu was afforded as described above. However, the lysine residue was protected in two different ways,.e.g., using Fmoc-(Lys)-(Mtt)-OH or Fmoc-(Lys)-(Alloc)-OH. This methodology allowed the synthesis of a cyclic hexapeptide in an end to side-chain fashion as well as a side-chain to side-chain fashion. For example, on-resin cyclization can be performed between the ε-amino group of Lys and the ω-carboxyl group of Glu in a side-chain to side-chain fashion as shown in Scheme 2 below to yield the cyclic peptide. Alternatively, cyclization can be performed between the N-α-amino terminus and the C-ω-carboxyl group of Glu in an end to side-chain fashion.

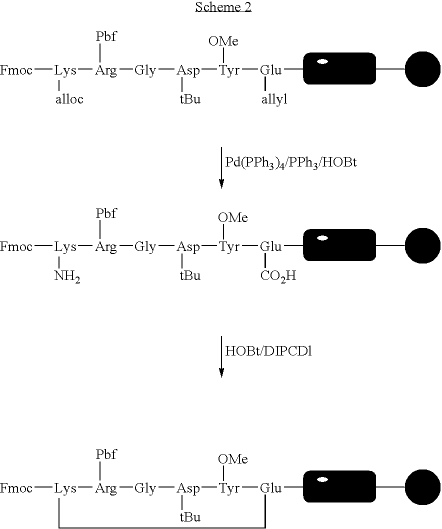

The peptides in Series III are pentapeptides containing the RGD motif and have the formula X-Lys-Arg-Gly-Asp. Series III peptides were synthesized starting from Fmoc-Asp-(PEG/PS)-(Oallyl), and cleavage from this resin yielded the peptide acid. The amino acid residue at position X was varied between the L- or D-forms of the hydrophobic Phe and Tyr residues. On-resin cyclization was performed between the amino and carboxyl termini of the peptide in an end to end fashion as shown in Scheme 3 below to yield the cyclic peptide.

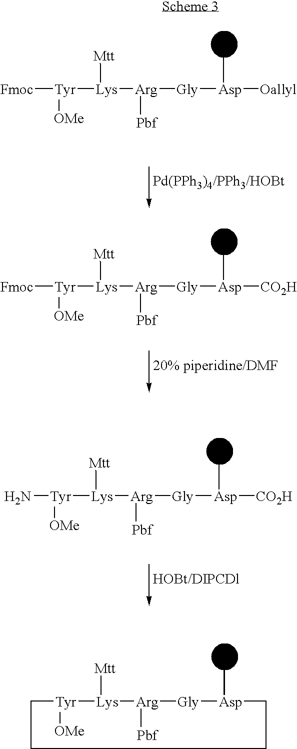

Linear peptides (A) were synthesized as follows. 0.5 g of the Fmoc-PAL-PEG/PS resin (resin loading 0.1-0.2 mmol/g) or Fmoc-(Asp)-(PEG/PS)-(Oallyl) (resin loading 0.17 mmol/g) was swollen for 1 hour. The resin was filtered and the Fmoc group was removed by shaking with 20% piperidine in DMF. The resin was filtered and washed with DMF, methanol, and DCM. A small sample of resin was taken and tested using the trinitrobenzene sulphonic acid (TNBS) method. When a positive result (i.e., red beads) was observed, the first amino acid was attached. The amino acids were used in 4-fold excess and acylation reactions were performed using DIPCDI (4 equivalents) and HOBt (4 equivalents). At the end of synthesis, the N-terminal Fmoc group was left intact. The peptidyl resin was washed with DMF, methanol, and DCM and dried under vacuum for 1 hour. A small sample was taken and small scale cleavage was performed (5-10 mg resin). The peptides were cleaved using 1.5 ml of a solution of TFA:water:TIPS at a ratio of 19:0.5:0.5 (v/v/v) for 1 hour. The resin was filtered through glass wool, washed with TFA, and the TFA was evaporated. The TFA was azeotroped with ether and the residue taken into water and washed with ether. The aqueous layer was freeze dried and subsequently analyzed using RP-HPLC and MALDI-TOF MS.

Cyclic peptides (B) were synthesized as follows. For the palladium-catalyzed removal of the allyl and alloc protecting groups, the linear peptidyl resins described above were swollen in DMF for 1 hour. All chemistry was performed in a glove box under an inert atmosphere. The peptidyl resins were washed with DMF and resuspended in fresh DMF. 10 equivalents of PPh₃ and HOBt were dissolved in DMF and added to the peptidyl resin. 0.25 g (1 equivalent) of Pd(PPh₃)₄ palladium catalyst was added and the reaction vessel wrapped in foil. Small samples were taken every hour for the first 8 hours and the reaction was left to proceed overnight (12-18 hours). The peptidyl resin samples were washed with 5% DIPEA and 5% diethyldithiocarbamate to remove excess palladium. The peptidyl resins were subsequently washed with copious amounts of DMF, methanol, and dichloromethane and vacuum dried for 1 hour. Small scale cleavage was performed using 1.5 ml of a solution of TFA:water:TIPS at a ratio of 19:0.5:0.5 (v/v/v) for 1 hour. The freeze dried samples were analyzed using RP-HPLC and MALDI-TOF MS.

On-resin cyclization was performed using DIPCDI (4 equivalents) and HOBt (4 equivalents). For all peptides except peptide 5 in Series II, the linear peptidyl resin was treated with 20% piperidine in DMF for 10 minutes after removal of the allyl group. The peptidyl resin was washed with DMF, methanol, and DCM and tested using the TNBS method. Once a positive result was observed, the cyclization reaction was started. For peptide 5, no piperidine treatment was required. Samples were taken every hour and ring closure was monitored using the TNBS method and RP-HPLC. Upon completion (i.e., a negative TNBS test), the resin was washed with DMF, methanol, and DCM and dried under vacuum. Small scale cleavage was performed using 1.5 ml of a solution of TFA:water:TIPS at a ratio of 19:0.5:0.5 (v/v/v) for 1 hour. The freeze dried samples were analyzed using RP-HPLC and MALDI-TOF MS.

Fluorobenzoyl cyclic peptides (C) were synthesized as follows. The ¹⁸F or ¹⁹F radionuclide was attached to a benzoyl group as shown in Scheme 4 below and the resulting 4-[¹⁸F]-fluorobenzoic acid or 4-[¹⁹F]-fluorobenzoic acid was selectively coupled to the peptidyl resin using HATU/DIPEA (4 equivalents) for 2 hours. The acylation reaction was monitored using the TNBS method. Upon completion (i.e., a negative TNBS test), the peptidyl resins were washed with DMF, methanol, and DCM and dried under vacuum. Small scale cleavage and analysis of the cyclic [¹⁸F]- or [¹⁹F]-fluorobenzoyl RGD peptides were performed as described above.

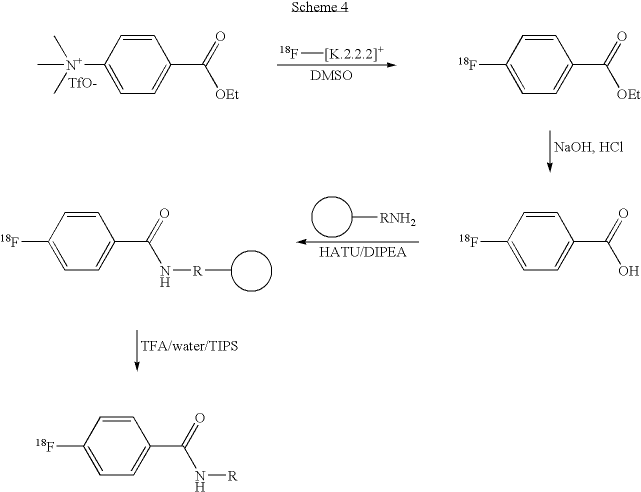

The fluorobenzoyl group was attached to either the ε-amino group or the α-amino group of Lys in the cyclic peptide. For conjugation to the ε-amino group of Lys, the Mtt protecting group was removed as follows (see, Scheme 5 below). A mixture of glacial acetic acid/trifluoroethanol/DCM at a ratio of 1:2:7 was added to the peptidyl resin for one hour. The peptidyl resin was washed with DMF, methanol, and DCM and a small amount tested using the TNBS method. Once a positive result was obtained, the peptidyl resin was swollen in DMF and acylated using 4-[¹⁸F]-fluorobenzoic acid or 4-[¹⁹F]-fluorobenzoic acid as described above. For conjugation to the a-amino group of Lys, the Fmoc group was removed as follows (see, Scheme 6 below). The peptidyl resin for peptide 5 was treated with 20% piperidine in DMF to yield the free amino terminus. Acylation was performed using using 4-[¹⁸F]-fluorobenzoic acid or 4-[19F]-fluorobenzoic acid as described above.

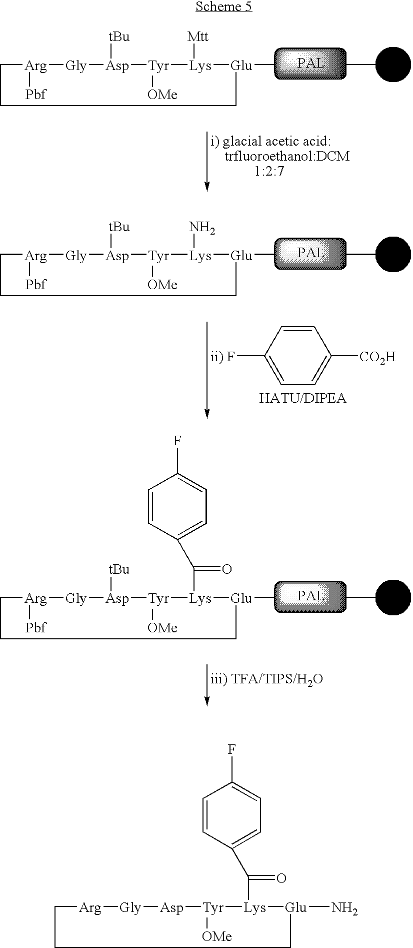

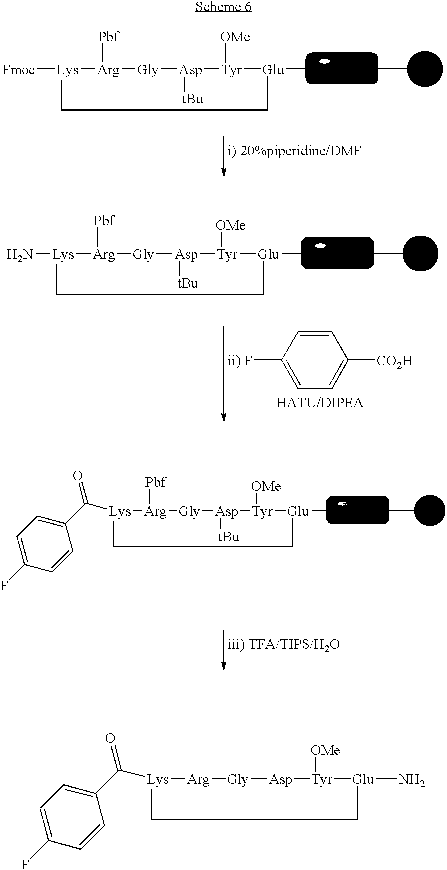

### Example 2

In vitro Analysis of RGD Peptides.

This example illustrates the in vitro biological activity and selectively of the RGD peptides synthesized in Example 1.

The receptor-binding affinity and selectivity of the RGD peptides of the present invention were assessed using the following methods: (1) assays to assess the ability of the peptides to inhibit adhesion of A375M and VUP cell lines to laminin and vitronectin substrates; and (2) enzyme-linked immunosorbent assays (ELISAS) using chimeric proteins comprising the extracellular domain of integrins linked to the Fc domain of IgG (Celltech plc; Slough, UK) to assess the affinity and selectively of the peptides towards a panel of immobilized RGD-binding integrins, e.g., αᵥβ₃, αᵥβ₅, α₅β₁, and α_{IIb}β₃.

### Materials and Methods

Reagents

Phosphate buffered saline (PBS), bovine serum albumin (BSA), Tween 20, tris[hydroxymethyl]aminomethane (Tris), and manganese chloride (MnCl₂) were purchased from Sigma (Dorset, UK). Sodium chloride was purchased from BDH (Dorset, UK). Tween 20 (protein grade) was purchased from Calbiochem (Nottingham, UK). Horseradish peroxidase-labelled F(ab)₂ fragment of goat anti-human IgG Fc or goat anti-murine Fc antibody was purchased from Jackson (Maine, USA). 3,3′, 5,5′-tetramethyl benzidene (TMB) was purchased from Intergen (Oxford, UK). Neutravidin-peroxidase was purchased from Pierce (Milwaukee, USA). A biotinylated fragment of vitronectin containing the RGD motif was supplied by IBMS (Southampton University, UK). A recombinant 50 kDa fragment containing the RGD domain of fibronectin was purified by Celltech. Fibrinogen was purchased from Sigma and biotinylated by Celltech plc. Positive peptide controls CT6483-69 (for αᵥβ₃ and αᵥβ₅) and CT7723-00 (for α₅β₁) were supplied by Celltech plc.

ELISA Analysis of RGD Peptides

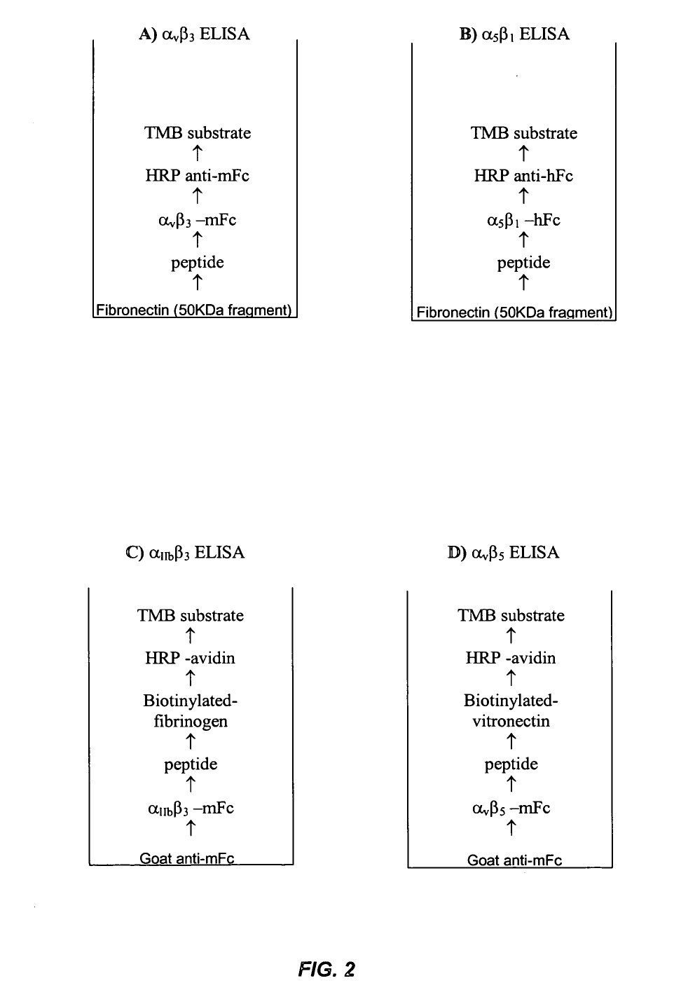

Four different soluble integrins were supplied by Celltech plc. Soluble forms of αᵥβ₃, αᵥβ₅, α₅β₁, and α_{IIb}β₃ integrin were generated by constructing chimeras comprising the extracellular domain of αᵥβ₃, αᵥβ₅, α₅β₁, and α_{IIb}β₃ linked to the Fc domain of either mouse IgG (α_{vβ}₃-mFc, α_{vβ}₅-mFc, and α_{IIbβ}₃-mFc) or human IgG (α₅β₁-hFc) as described by Stephens et al., Cell Adhes. Commun., 7:377-390 (2000) and Coe et al., J. Biol. Chem., 276:35854-35866 (2001). Since the four soluble integrin chimeras were supplied either as purified proteins (αᵥβ₃-mFc and α₅β₁-hFc) or as unpurified hybridoma supernatants (α_{IIb}β₃-mFc and αᵥβ₅-mFc), it was necessary to use two types of ELISAs. FIG. 2 shows the type of ELISA performed for each of the four integrins. In one type of ELISA (FIGS. 2A and 2B), the immobilized component is a 50 kDa fragment of fibronectin. In the second type of ELISA (FIGS. 2C and 2D), the immobilized component is a goat anti-mFc antibody that is used to capture chimeric proteins so that the RGD binding site of the integrin is maximally exposed. Each three-sided box in FIG. 2 represents a single well of a 96-well plate, and the components are listed in the sequence of their addition to the assay. The immobilized component at the bottom of the well is bound to the well by electrostatic interactions.

To analyze the affinity and selectively of RGD peptides for αᵥβ₃ and α₅β₁, 96-well ELISA plates were coated with 100 μl of a 5 μg/ml of the 50 kDa fragment of fibronectin in PBS per well and left overnight at 4° C. The plates were washed with PBS using a Denley Wellwash 4 Mk 2. Washing was repeated twice with a 400 μl wash per well. 200 μ/well of blocking buffer (i.e., to prevent non-specific binding of proteins) was then added for 1 hour followed by a repeat washing with PBS. RGD peptides were then added at a maximum concentration of 200 μM and a preliminary screen was performed at three concentrations, 200,20, and 2 μM. 100 μl of an RGD peptide was added to each well and each assay was performed in triplicate. Purified soluble αᵥβ₃-mFc or purified α₅β₁-hFc was diluted to 2 μg/ml and 15 ng/ml, respectively, in conjugate buffer and 100 μl was added to each well. The plates were incubated for 2 hours with shaking using a Luckham R100 Rotatest at approximately 150 rpm. After incubation, the plates were washed with PBS as previously described and a labeled antibody was added. For the αᵥβ₃ assay, a horseradish peroxidase (HRP)-labeled F(ab′)₂ fragment of goat anti-mouse IgG Fc was diluted 1:2000 in conjugate buffer and 100 μl was added to each well. For the α₅β₁ assay, an HRP-labeled F(ab′)₂ fragment of goat anti-human IgG Fc was diluted 1:2000 in conjugate buffer and 100 μl was added to each well. Plates were incubated for 30 minutes with shaking followed by 2 washes with PBS. 100 μl of TMB substrate was added to each well and plates were shaken during a 10 minute color development. Plates were read using a plate spectrophotometer at 630 nm.

To analyze the affinity and selectively of RGD peptides for α_{IIb}β₃ and αᵥβ₅, 96-well ELISA plates were coated with anti-murine Fc antibody at 5 μg/ml in PBS, 100 μl per well and left overnight at 4° C. The plates were washed with PBS using a Denley Wellwash 4 Mk 2. Washing was repeated twice with a 400 μl wash per well. 200 μl/well of blocking buffer was then added for 1 hour followed by a repeat washing with PBS. 100 μl of tissue culture supernatant from cells secreting soluble α_{IIb}β₃-mFc or αᵥβ₅-mFc, diluted 1:2 in conjugate buffer, was added to each well. Plates were incubated with shaking for 1 hour followed by two washes with PBS. 100 μl of an RGD peptide (at concentrations of 200, 20, and 2 μM), followed by either 100 μl of biotinylated fibrinogen (at 1 μg/ml in conjugate buffer) or 100 μl of biotinylated vitronectin (at 2 μg/ml in conjugate buffer) were added to each well and incubated for 2 hours with shaking. After incubation, the plates were washed with PBS as described above. 100 μl of neutravidin-peroxidase was added to each well and the plates were incubated for 30 minutes with shaking, followed by two washes with PBS. 100 μl of TMB substrate was added to each well and the plates were shaken during color development. Plates were read using a plate spectrophotometer at 630 nm.

Two negative and two positive controls were used in both types of ELISAs. For example, the negative controls were wells that contained 50 μl of an active control peptide, integrin, and biotinylated ligand or 50 μl of the active control peptide and 50 μl of buffer. These negative control wells typically develop little to no color. The positive controls were wells that did not contain the active control peptide but did contain integrin and biotinylated ligand or 50 μl of an irrelevant peptide, integrin, and biotinylated ligand. These wells typically develop the maximum amount of color.

Adhesion Assay Analysis of RGD Peptides

RGD peptides were plated at a maximum concentration of 200 μM and a minimum concentration of 2 nM. For each RGD peptide sequence, linear peptides (A), cyclic peptides (B), and 4-[¹⁹F]-fluorobenzoyl cyclic peptides (C) were assayed. Each RGD peptide concentration was performed in quadruplicate and each experiment was repeated twice.

Plastic 96-well plates (Falcon 3912; Becton Dickinson) were coated with 50 μl substrate (10 μg/ml laminin or 5 μg/ml vitronectin). The plates were incubated at 37° C. in an 8% CO₂ atmosphere for 1 hour. Unbound protein was flicked off and the plates were washed with phosphate buffered saline (PBS) and blocked with bovine serum albumin (0.1% w/v BSA)/PBS/0.1% sodium azide) for 1 hour. The plates were then washed with PBS, placed on a bed of ice and 25 μl of peptide was added to the wells.

Sodium [⁵¹Cr] chromate was purchased from Amersham International, UK. The solution was made isotonic by the addition of 110 μl 10× Hanks buffered salt solution. 100 μl of this solution (3.7 MBq) was added to 5-10×10⁶ cells in 500 μl of serum-containing growth medium. The suspension was incubated at 37° C. for 45 minutes with regular agitation to resuspend the cells. The cells were then washed and spun 3 times with serum-free E4 medium to remove any free [⁵¹Cr]chromium. A trypan blue viability count was performed and the cells were diluted to the necessary volume in serum-free E4 medium (4×10⁵ cells/ml).

25 μl (about 10,000 cells) were added to each quadruplicate well and the plates incubated at 37° C. in an 8% CO₂ atmosphere for 1 hour. Unbound cells were flicked off and the plates were washed twice with PBS/BSA containing 1 mM CaCl₂ and 0.5 mM MgCl₂. The plates were dabbed dry and cut into individual wells. The radioactivity associated with each well was determined in a gamma counter (1261 Multigamma; Wallac, Sweden). Quadruplicate 25 μl samples of labeled cells were counted as 100% input values. To determine non-specific binding, the ability of cells to bind BSA-coated wells was also assessed. Data for these wells are referred to as background.

The results were calculated as follows:

$$\% Adhesion = \frac{mean cpm of substrate wells - mean cpm of BSA wells}{input counts} \times 100$$

$$Standard deviation ( SD ) of \% adhesion = \frac{SD of mean cpm of substrate wells}{mean cpm of substrate wells} \times \% adhesion$$

NMR Spectroscopy of RGD Peptides

In order to determine whether peptide biological behavior could be correlated to peptide structure, nuclear magnetic resonance (NMR) spectroscopy was applied to probe the structural characteristics of specific RGD peptides. NMR spectroscopy has the ability to view peptides in solution at the atomic level and distinguish individual atoms within their specific chemical and structural environments. All NMR spectroscopy experiments were ¹H (proton) NMR spectroscopy experiments, where ¹H atoms within the peptide sample of interest were detected. The specific details of NMR spectroscopy theory and operation are known to those skilled in the art and are reviewed in, e.g., Wuthrich, NMR of Protein and Nucliec Acids (1986); Cavanagh et al., Protein NMR Spectrocopy: Principles and Practice, Academic Press (1996); Howard, Curr. Biol., May 7;8(10):R331-3 (1998).

The NMR spectroscopy experiments conducted were total correlation spectroscopy (TOCSY) (Braunschweiler et al., J. of Magnetic Resonance, 53:521-528 (1983)) and nuclear Overhauser effect spectroscopy (NOESY) (Jeener et al., J. of Chemistry and Physics, 71:4553 (1979)). The TOCSY experiment identifies and collates ¹H atoms from each amino acid in a peptide. The NOESY experiment identifies pairs of ¹H atoms that are close in space (e.g., within 6 Å) due to conformation or structural folds. It is the NOESY experiment that can be used to build structural models of peptides and proteins in solution. For example, if a particular conformation or structure is held by a peptide, one expects to observe NOESY contacts between amino acids that are not adjacent in sequence (i.e., contact residues that are more than one amino acid apart in sequence). Adjacent NOESY contacts are described as (i-i+1) sequential contacts and non-adjacent contacts are described as i-i+2, i-i+3, etc.

NMR spectroscopy data were obtained from a Varian Unity INOVA 600 MHz NMR spectrometer operating at 10° C. Samples were dissolved in 600 μl of buffer (25 mM PBS at pH 6.4, 100 mM sodium chloride) and placed in a Wilmad 535-PP7 5 mm NMR tube for detection. NMR spectroscopy data were then collected and analyzed. RGD peptides A1, B1, C1, B7, and C7 were analyzed by NMR spectroscopy as described below.

### Results

ELISA Analysis of RGD Peptides

8 out of the 10 peptide sequences synthesized in Example 1 were assayed by ELISA. Each peptide was tested in a preliminary screen as a linear peptide (A), a cyclic peptide (B), and a 4-[¹⁹F]-fluorobenzoyl cyclic peptide (C). For example, Al refers to the linear form of peptide #1 in FIG. 1, B1 refers to the cyclic form, and C1 refers to the 4-[¹⁹F]-fluorobenzoyl cyclic form. A11 refers to the linear peptide H-KPQVTRGDVFTEG-NH₂. From this screen, promising 4-[¹⁹F]-fluorobenzoyl cyclic peptides, e.g., those with increased receptor affinity and/or selectively, were identified and further investigated.

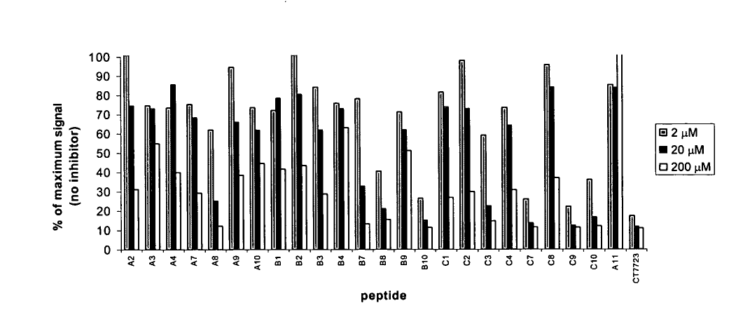

FIGS. 3-6 illustrate the percent binding of the vitronectin, fibronectin, or fibrinogen ligand to αᵥβ₅, α₅β₁, α_{IIb}β₃, or αᵥβ₃ integrin in the presence of RGD peptides (i.e., linear (A), cyclic (B), or 4-[¹⁹F]-fluorobenzoyl cyclic (C)) at different concentrations, with 100% maximum signal being the signal obtained in the absence of the peptide.

FIG. 3 shows that, with the exception of A8, the linear RGD peptides tested (i.e., A2, A3, A4, A7, A9, and A10) had little effect on αᵥβ₅ binding to vitronectin at concentrations of 2 μM and 20 μM, and were only capable of inhibiting greater than about 50% of the interaction between αᵥβ₅ and vitronectin at the highest concentration (200 μM). 20 μM of linear peptide A8 inhibited the interaction between αᵥβ₅ and vitronectin to 25.3% of the maximum signal. FIG. 3 also shows that cyclization of the linear peptides can improve their inhibitory efficacy. For example, 20 μM of cyclic peptide B7 (i.e., the cyclized form of A7) inhibited the interaction between αᵥβ₅ and vitronectin to 32.8% of the maximum signal and 2 μM of cyclic peptide B10 (i.e., the cyclized form of Al0) inhibited the interaction between αᵥβ₅ and vitronectin to 26.7% of the maximum signal. Addition of the 4-[¹⁹F]-fluorobenzoyl moiety had a further effect on the inhibiting properties of all the cyclic RGD peptides except for B8 and B10. For example, a significant inhibitory effect was observed at only 2 μM of C7 (i.e., the 4-[¹⁹F]-fluorobenzoyl form of B7), which reduced vitronectin binding to 26.1% the maximum signal. Similarly, a significant inhibitory effect was observed at only 2 μM of C9 (i.e., the 4-[¹⁹F]-fluorobenzoyl form of B9), which reduced vitronectin binding to 22.2% of the maximum signal. However, addition of the 4-[¹⁹F]-fluorobenzoyl moiety to B8 to create C8 had the inverse effect, resulting in an increased amount of binding between vitronectin and αᵥβ₅, i.e., from 21.2% for B8 to 84.2% for C8, at 20 μM. Linear peptide A11 had no effect on the binding of vitronectin to αᵥβ₅.

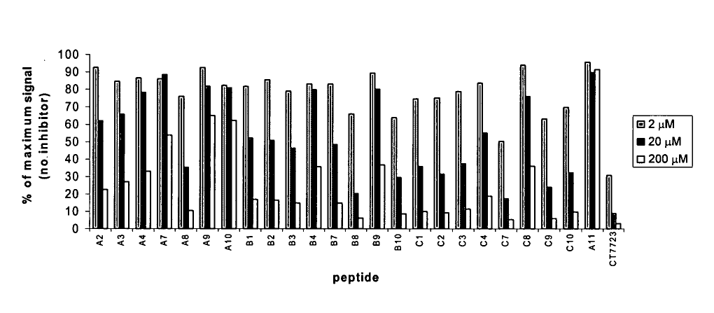

FIG. 4 shows that, with the exception of A8, the linear RGD peptides tested (i.e., A2, A3, A4, A7, A9, and A10) had little effect on α₅β₁, binding to fibronectin at concentrations of 2 μM and 20 μM. FIG. 4 also shows that A2, A3, and A4 were only capable of inhibiting greater than about 50% of the interaction between α₅β₁ and fibronectin at the highest concentration (200 μM), while A7, A9, and A10 were less than 50% effective even at the highest concentration. 20 μM of linear peptide A8 inhibited the interaction between α₅β₁ and fibronectin to 35.4% of the maximum signal. Further, FIG. 3 shows that cyclization of the linear peptides can improve their inhibitory efficacy. For example, 20 μM of cyclic peptide B7 (i.e., the cyclized form of A7) inhibited the interaction between α₅β₁ and fibronectin to 48.4% of the maximum signal and 20 μM of cyclic peptide B10 (i.e., the cyclized form of A10) inhibited the interaction between α₅β₁ and fibronectin to 29.6% of the maximum signal. Addition of the 4-[¹⁹F]-fluorobenzoyl moiety had a further effect on the inhibiting properties of all the cyclic RGD peptides except for B8 and B10. However, addition of the 4-[¹⁹F]-fluorobenzoyl moiety to B8 to create C8 had the inverse effect, resulting in an increased amount of binding between fibronectin and α₅β₁, i.e., from 22% for B8 to 79.8% for C8, at 20 μM. Linear peptide A11 had no effect on the binding of fibronectin to α₅β₁.

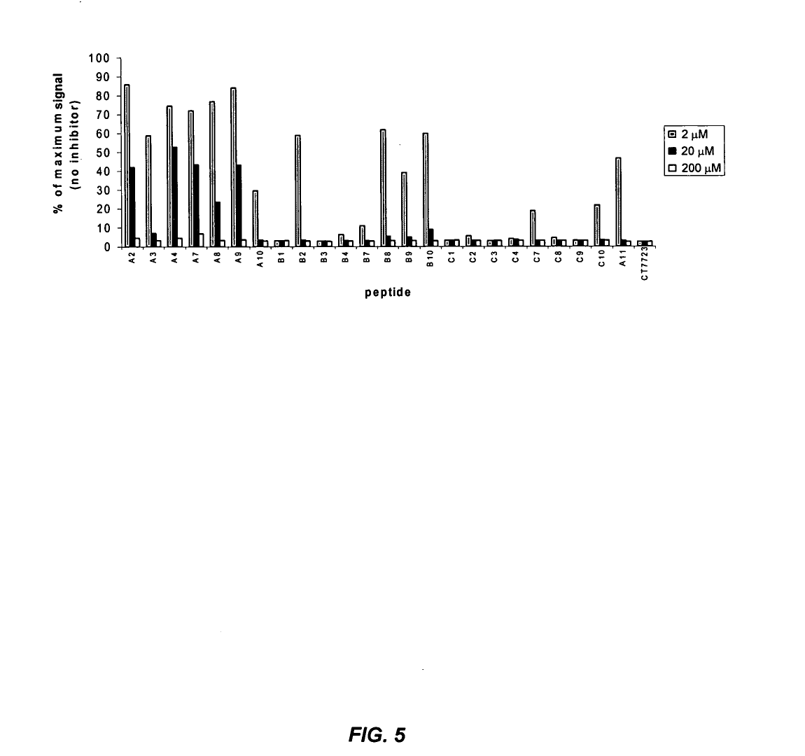

FIG. 5 shows that all of the linear RGD peptides tested (i.e., A2, A3, A4, A7, A8, A9, and A10) inhibited greater than about 50% of the interaction between α_{IIb}β₃ and fibrinogen at 20 μM. In fact, A10 was capable of inhibiting the interaction between α_{IIb}β₃ and fibrinogen to 29.6% even at the lowest concentration (2 μM). FIG. 5 also shows that, with the exception of B10, cyclization of the linear peptides further enhanced their inhibitory effect by significantly reducing fibrinogen binding to α_{IIb}β₃. Addition of the 4-[¹⁹F]-fluorobenzoyl moiety had a further effect on the inhibiting properties of the cyclic RGD peptides B2, B8, B9, and B10. For example, a significant inhibitory effect was observed at only 2 μM of C2 (i.e., the 4-[¹⁹F]-fluorobenzoyl form of B2), which reduced fibrinogen binding to 5.7% the maximum signal. Similarly, a significant inhibitory effect was observed at only 2 μM of C8 (i.e., the 4-[¹⁹F]-fluorobenzoyl form of B8), which reduced fibrinogen binding to 4.6% the maximum signal. Likewise, a significant inhibitory effect was observed at only 2 μM of C9 (i.e., the 4-[¹⁹F]-fluorobenzoyl form of B9), which reduced fibrinogen binding to 3.2% the maximum signal. A significant inhibitory effect was also observed at only 2 μM of C10 (i.e., the 4-[¹⁹F]-fluorobenzoyl form of B10), which reduced fibrinogen binding to 21.9% the maximum signal. Linear peptide A11 had a significant effect on the binding of fibrinogen to α_{IIb}β₃ 20 μM.

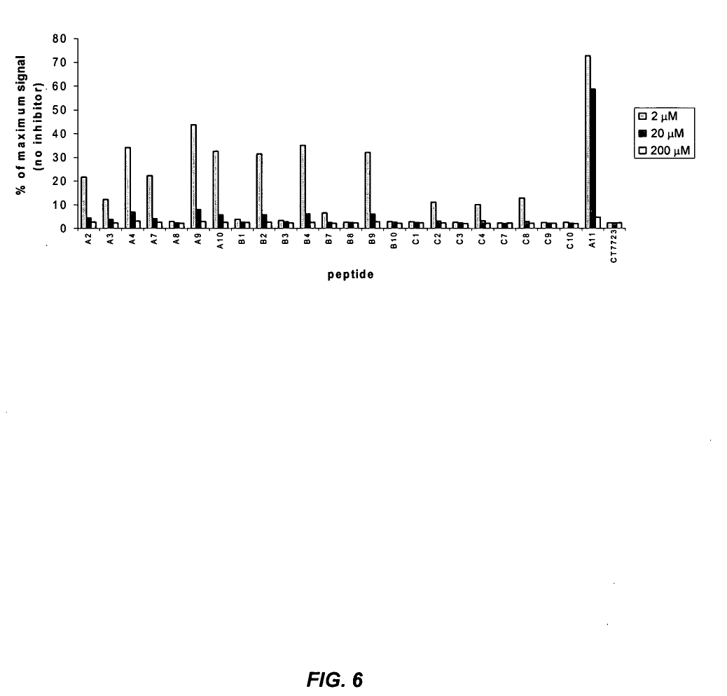

FIG. 6 shows that all of the linear RGD peptides tested (i.e., A2, A3, A4, A7, A8, A9, and A10) inhibited greater than about 50% of the interaction between αᵥβ₃ and fibronectin at all concentrations. FIG. 6 also shows that, with the exception of B2 and B4, cyclization of the linear peptides further enhanced their inhibitory effect by significantly reducing fibronectin binding to αᵥβ₃. For example, 2 μM of cyclic peptide B3 (i.e., the cyclized form of A3) inhibited the interaction between αᵥβ₃ and fibronectin to 3.5% of the maximum signal; 2 μM of cyclic peptide B7 (i.e., the cyclized form of A7) inhibited the interaction between αᵥβ₃ and fibronectin to 6.6% of the maximum signal; 2 μM of cyclic peptide B9 (i.e., the cyclized form of A9) inhibited the interaction between αᵥβ₃ and fibronectin to 32.1 % of the maximum signal; and 2 μM of cyclic peptide B10 (i.e., the cyclized form of A10) inhibited the interaction between αᵥβ₃ and fibronectin to 3.0% of the maximum signal. Addition of the 4-[¹⁹F]-fluorobenzoyl moiety had a further effect on the inhibiting properties of the cyclic RGD peptides B2, B4, and B9. For example, a significant inhibitory effect was observed at only 2 μM of C2 (i.e., the 4-[¹⁹F]-fluorobenzoyl form of B2), which reduced fibronectin binding to 11.1% of the maximum signal. Similar results were obtained for C4 and C9 (i.e., the 4-[¹⁹F]-fluorobenzoyl forms of B4 and B9), which reduced fibronectin binding to 10.2% and 2.6% of the maximum signal, respectively. Linear peptide A11 had little effect at 2 μM but inhibited fibronectin binding to 58.7% of the maximum signal at 20 μM.

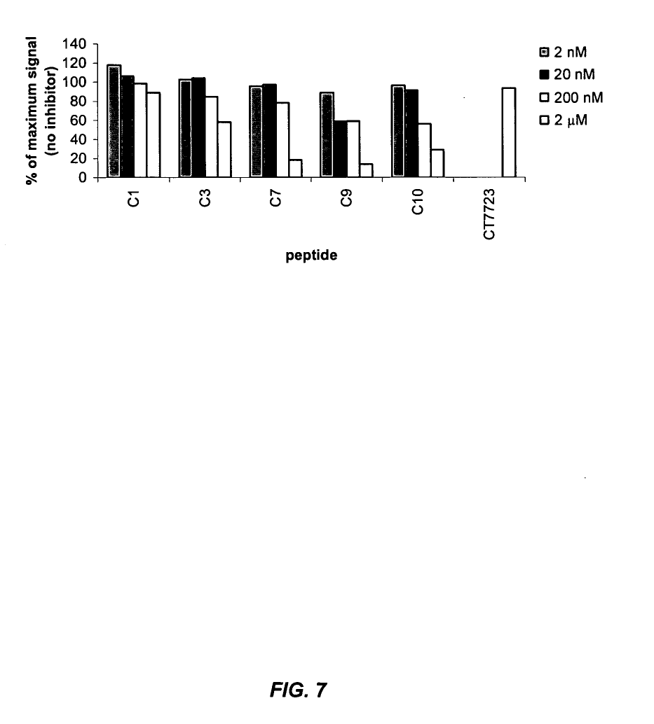

The most promising 4-[¹⁹F]-fluorobenzoyl cyclic peptides, i.e., C1, C3, C7, C9, and C10, were selected and their ability to inhibit the binding of the vitronectin, fibronectin, or fibrinogen ligand to αᵥβ₅, α₅β₁, α_{IIb}β₃, or αᵥβ₃ integrin was assayed over a concentration range of from 2 nM to 20 μM. The results are shown in FIGS. 7-10.

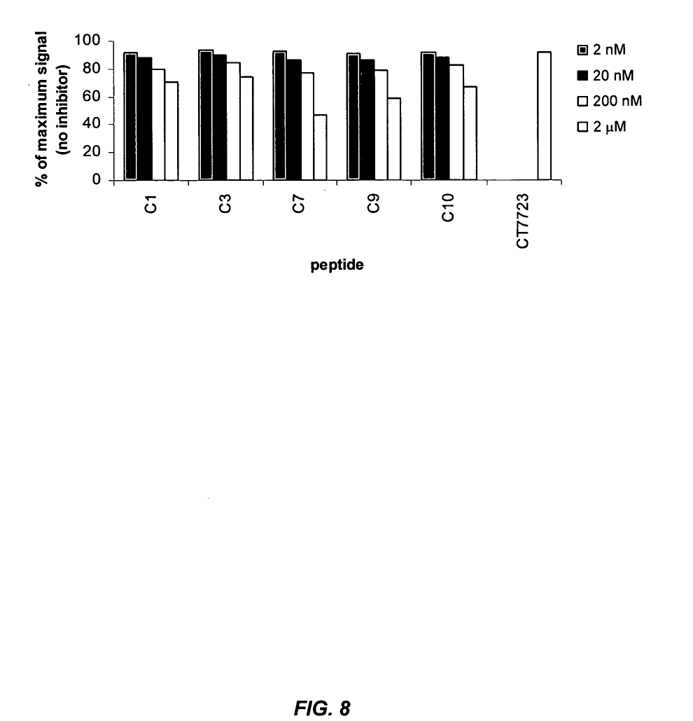

FIG. 7 shows the inhibitory effect of C1, C3, C7, C9, and C10 on the binding between αᵥβ₅ and vitronectin at concentrations of 2 nM, 20 nM, 200 nM, and 20 μM. At nanomolar concentrations, none of these peptides had a significant effect on the binding of vitronectin to αᵥβ₅. The maximum inhibitory effect was observed with C9, which reduced vitronectin binding to 58.9% of the maximum signal at 20 nM. FIG. 8 shows that none of these peptides had a significant effect on α₅β₁ binding to fibronectin. Even at micromolar concentrations (2 μM), the maximum inhibitory effect observed only reduced fibronectin binding to 47% of the maximum signal (see, peptide C7 in FIG. 8).

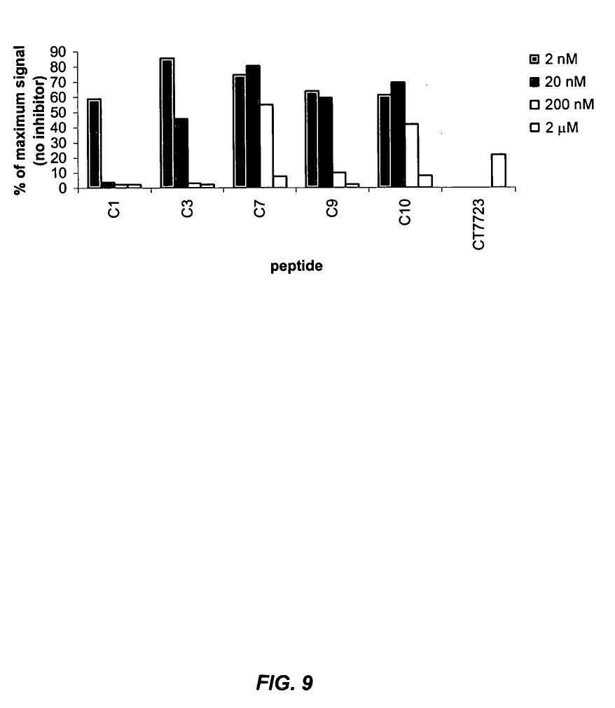

FIG. 9 shows that peptides C1, C3, and C9 had a significant effect on the binding of α_{IIb}β₃ to fibrinogen at nanomolar concentrations. The most significant inhibitory effect was observed with C1, which reduced fibrinogen binding to 59% at 2 nM. Peptides C3 and C9 also showed a significant inhibitory effect at 20 nM, reducing fibrinogen binding to 45.8% and 59.5%, respectively. However, peptides C7 and C10 did not effectively block the binding of α_{IIb}β₃ to fibrinogen at nanomolar concentrations, as fibrinogen binding was still 55% and 42.1%, respectively at 200 nM.

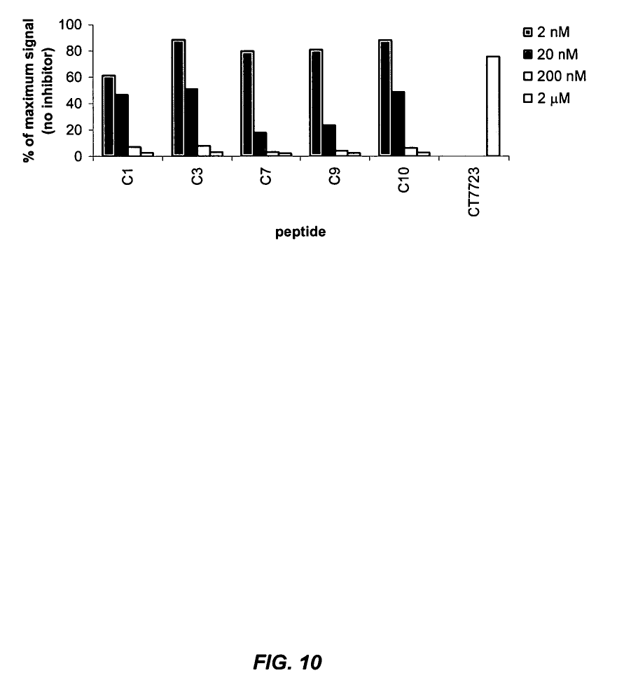

FIG. 10 shows that all peptides significantly inhibited the binding of αᵥβ₃ to the 50 kDa fibronectin fragment at nanomolar concentrations. Peptides C7 and C9 had the greatest inhibitory effect at 20 nM, reducing fibronectin binding to 18% and 23.5%, respectively.

IC₅₀ Analysis of RGD Peptides C7 and C10

The IC₅₀ values were calculated for peptides C7 and C10. C7 was selected due to its striking selectively for inhibiting αᵥβ₃ binding at nanomolar concentrations, as it had little effect on the binding of the other three integrins at such low concentrations. C10 was selected due to its selectively for inhibiting αᵥβ₃ binding at nanomolar concentrations, as compared to its less pronounced effect on the binding of the other three integrins at such low concentrations. Although C9 was more effective than C10 at inhibiting αᵥβ₃ binding, it also had a greater effect at inhibiting α_{IIb}β₃ binding than C7 or C1O and was excluded from the IC₅₀ analysis. However, C9 was capable of selectively inhibiting αᵥβ₃ integrin binding at 20 nM.

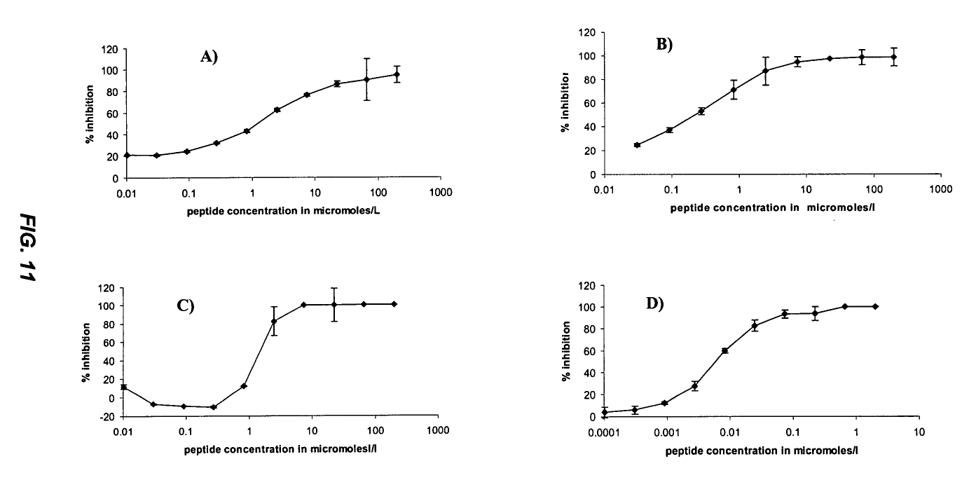

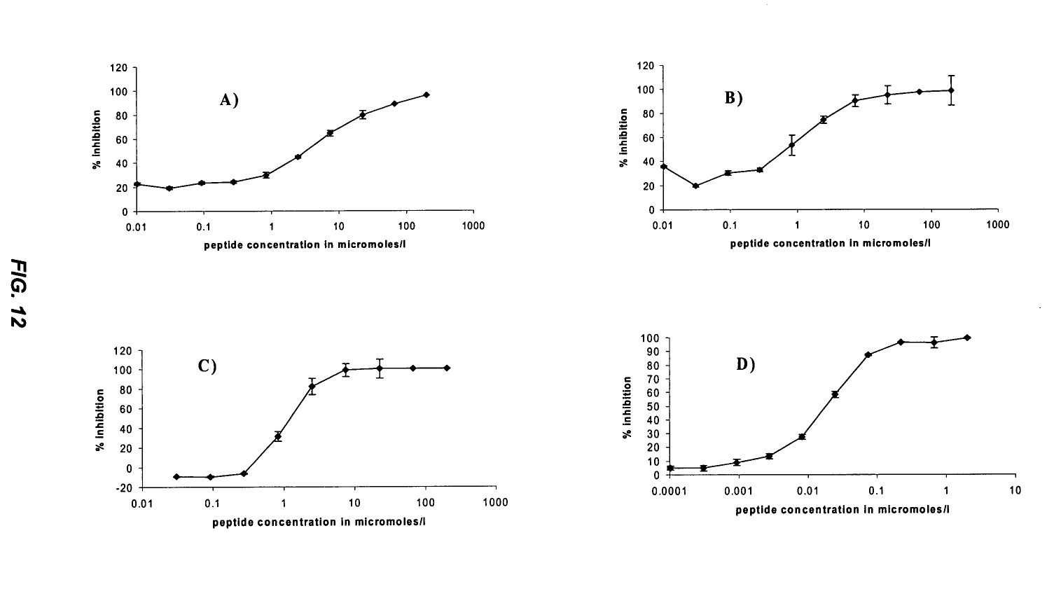

Peptides C7 and C10 were titrated in triplicate dilution from 200 μM to 0.02 μM for all integrins except for αᵥβ₃, in which the titration started at 2 μm and ended at 0.2 nM. FIGS. 11 and 12 show the inhibitory effects of peptides C7 and C10 on: A) αᵥβ₅; B) α₅β₁; C) α_{IIb}β₃; and D) αᵥβ₃. Mean IC₅₀ values were calculated and are shown in Table 2 below. Peptide C7 was found to have an IC₅₀ value of 6.22 nM for αᵥβ₃, 481 nM for α₅β₁, 1.52 μM for α_{IIb}β₃, and 1.69 μM for αᵥβ₅. The IC₅₀ values α₅β₁, α_{IIb}β₃, and αᵥβ₅ were 77, 244, and 271 fold lower, respectively, than the value obtained for αᵥβ₃. These data suggest that C7 is a highly selective and potent inhibitor of αᵥβ₃ integrin.

TABLE 2
Mean IC₅₀ values for peptides C7 and C10.

| | IC₅₀ | Compared | IC₅₀ | Compared |
|---|---|---|---|---|
| Integrin | C7 | with αᵥβ₃ | C10 | with αᵥβ₃ |
| αᵥβ₅ | 1.69 | 271 | 6.01 | 328 |
| α₅β₁ | 0.48 | 77 | 1.37 | 75 |
| α_{II}β₃ | 1.52 | 244 | 1.23 | 67 |
| αᵥβ₃ | 0.006 | | 0.018 | |

Adhesion Assay Analysis of RGD Peptides

Two sets of peptides, A7, B7, C7 and A10, B10, C10, were titrated in adhesion assays. The A375M and VUP melanoma cell lines were used with vitronectin and laminin as substrates. Peptides were titrated in 10-fold dilutions from 200 μM to 2 nM. All data were normalized to the absence of peptide, which corresponded to 100% adhesion. All titrations were performed in quadruplicate and each experiment was repeated at least twice.

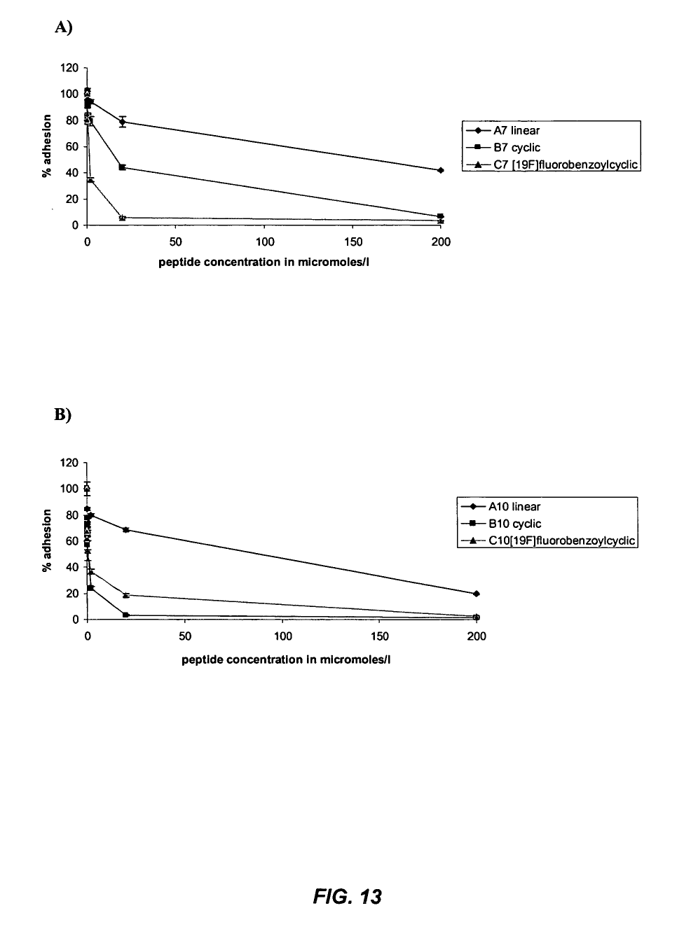

FIG. 13A shows the effect of A7, B7, and C7 on the binding of [⁵¹Cr]-VUP cells to vitronectin. Initial binding in the absence of peptide was 32.96%±4.58%. The graph shows that the linear version of the peptide (A7) had the least effect on inhibiting cell binding, as it only reduced adhesion to 78.65%±3.98% at 20 μM. The cyclic version of the peptide (B7) had a more pronounced effect on inhibiting cell adhesion, as it reduced adhesion to 44.16%±1.71% at 20 μM. The 4-[¹⁹F]-fluorobenzoyl cyclic version of the peptide (C7) had the greatest effect on inhibiting cell adhesion, as it reduced adhesion to 5.95%±0.39% at 20 μM. As such, the addition of a 4-[¹⁹F]-fluorobenzoyl moiety on B7 significantly increased its potency for inhibiting cell adhesion to an RGD-containing substrate.

FIG. 13B shows the effect of A10, B10, and C10 on the binding of [⁵¹Cr]-VUP cells to vitronectin. Initial binding in the absence of peptide was 14.31%±0.09%. The graph shows that the linear version of the peptide (A10) had the least effect on inhibiting cell binding, as it only reduced adhesion to 68.79%±1.51% at 20 μM. The cyclic version of the peptide (B10) had the greatest effect on inhibiting cell adhesion, as it reduced adhesion to 3.37%±0.53% at 20 μM. The 4-[¹⁹F]-fluorobenzoyl cyclic version of the peptide (C10) also had a significant effect on inhibiting cell adhesion, as it reduced adhesion to 18.64%±1.23% at 20 μM.

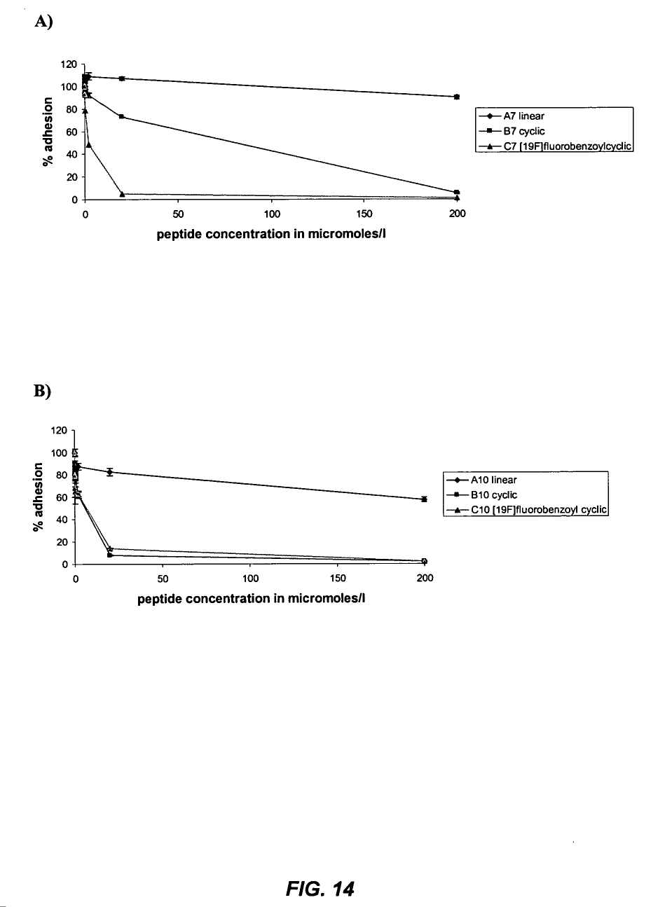

FIG. 14A shows the effect of A7, B7, and C7 on the binding of [⁵¹Cr]-A375M cells to vitronectin. Initial binding in the absence of peptide was 40.52%±4.56%. The graph shows that the linear version of the peptide (A7) had the least effect on inhibiting cell binding, as cell adhesion in the presence of A7 was 106.99±1.66% at 20 μM. The cyclic version of the peptide (B7) had a more pronounced effect on inhibiting cell adhesion, as it reduced adhesion to 72.79%±0.04% at 20 μM. The 4-[¹⁹F]-fluorobenzoyl cyclic version of the peptide (C7) had the greatest effect on inhibiting cell adhesion, as it reduced adhesion to 4.95±57% at 20 μM. As such, the addition of a 4-[¹⁹F]-fluorobenzoyl moiety on B7 significantly increased its potency for inhibiting cell adhesion to an RGD-containing substrate. In particular, C7 was about 100 fold better than B7 at inhibiting adhesion of [⁵¹Cr]-A375M cells to vitronectin (i.e., 72.79%±0.04% at 20 μM for B7 versus 78.87%±2.71% at 200 nM for C7).

FIG. 14B shows the effect of A10, B10, and C10 on the binding of [⁵¹Cr]-A375M cells to vitronectin. Initial binding in the absence of peptide was 32.94%±1.65%. The graph shows that the linear version of the peptide (A10) had the least effect on inhibiting cell binding, as cell adhesion in the presence of A10 was 82.58%±3.48% at 20 μM. The cyclic version of the peptide (B10) and the 4-[¹⁹F]-fluorobenzoyl cyclic version of the peptide (C10) had similar effects on inhibiting cell binding, as they reduced adhesion to 7.74%±0.42% and 13.89%±0.21%, respectively, at 20 μM.

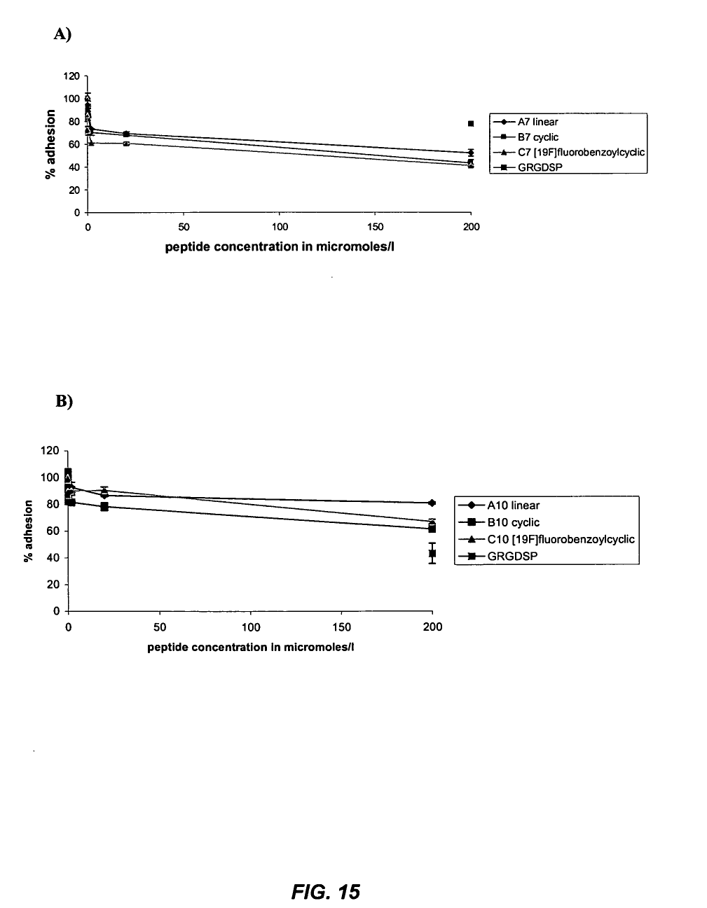

FIG. 15A shows the effect of A7, B7, and C7 on the binding of [⁵¹Cr]-VUP cells to laminin. Initial binding in the absence of peptide was 20.56%±2.95%. All three peptides had a similar effect on inhibiting cell binding, as cell adhesion in the presence of A7, B7, and C7 was 64.17%±1.31%, 46.89%±1.45%, and 45.38%±1.97%, respectively at the highest concentration (200 μM). The control peptide having the sequence GRGDSP had a similar inhibitory effect on cell adhesion (49.29%±2.74%).

FIG. 15B shows the effect of A10, B10, and C10 on the binding of [⁵¹Cr]-VUP cells to laminin. Initial binding in the absence of peptide was 32.54%±2.68%. All three peptides had a similar effect on inhibiting cell binding, as cell adhesion in the presence of A10, B10, and C10 was 80.67%±0.86%, 61.23%±2.06%, and 66.83%±1.62%, respectively at the highest concentration (200 μM). The control peptide having the sequence GRGDSP had a similar inhibitory effect on cell adhesion (43.05%±7.66%).

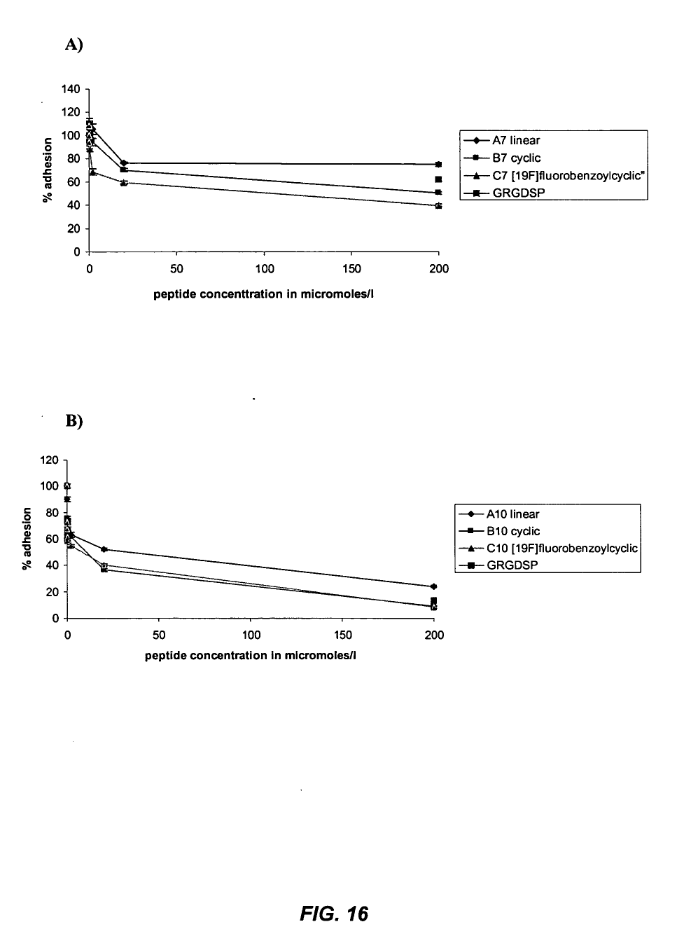

FIG. 16A shows the effect of A7, B7, and C7 on the binding of [⁵¹Cr]-A375M cells to laminin. Initial binding in the absence of peptide was 28.92%±3.06%. All three peptides only had a similar effect on inhibiting cell binding, as cell adhesion in the presence of A7, B7, and C7 was 75.08%±1.53%, 50.40%±1.24%, and 39.81%±1.11%, respectively at the highest concentration (200 μM). The control peptide having the sequence GRGDSP had a similar inhibitory effect on cell adhesion (62.05%±1.88%).

FIG. 16B shows the effect of A10, B10, and C10 on the binding of [⁵¹Cr]-A375M cells to laminin. Initial binding in the absence of peptide was 36.17%±3.62%. All three peptides had a similar effect on inhibiting cell binding, as cell adhesion in the presence of A10, B10, and C10 was 63.10%±2.01%, 61.88%±0.75%, and 54.76%±1.072%, respectively at only 2 μM. The control peptide having the sequence GRGDSP also had an inhibitory effect on cell adhesion (13.51%±1.25% at 200 μM).

Taken together, the ELISA and adhesion assay analysis indicate that peptide C7, having the sequence 4-[¹⁹F]-fluorobenzoyl cyclic (RGDY(OMe)K), in which the 4-[¹⁹F]-fluorobenzoyl moiety is conjugated to the ε-amino group of K, is a high affinity and selective inhibitor of αᵥβ₃ integrin. The results from these assays also indicate that the addition of a fluorobenzoyl moiety to the cyclic peptide further increases the potency and selectively of the peptide.

NMR Spectroscopy of RGD Peptides

To assess whether the increase in affinity and selectively upon the addition of a fluorobenzoyl moiety to B7 was due to structural modifications, the NMR spectra of B7 and C7 were compared. Because pentapeptides are thought to be more selective than hexapeptides towards αᵥβ₃ integrin (Gurrath et al., Eur. J. Biochem., 210:911-921 (1992)), peptides A1, B1, and C1, a set of hexapeptides having the sequence (RGDY(OMe)KE), were also analyzed by NMR spectroscopy.

The TOCSY spectra for A1, B1 and C1 indicated that a single conformation was present in all of these peptides and the NOESY data for A1 and B1 contained only i-i+1 NOESY contacts, thus showing no evidence of any structural conformation being held by these peptides. The addition of the 4-[¹⁹F]-fluorobenzoyl moiety in C1 did not change the structural characteristics of C1 when compared with B1. In fact, both B1 and C1 were equally unstructured.

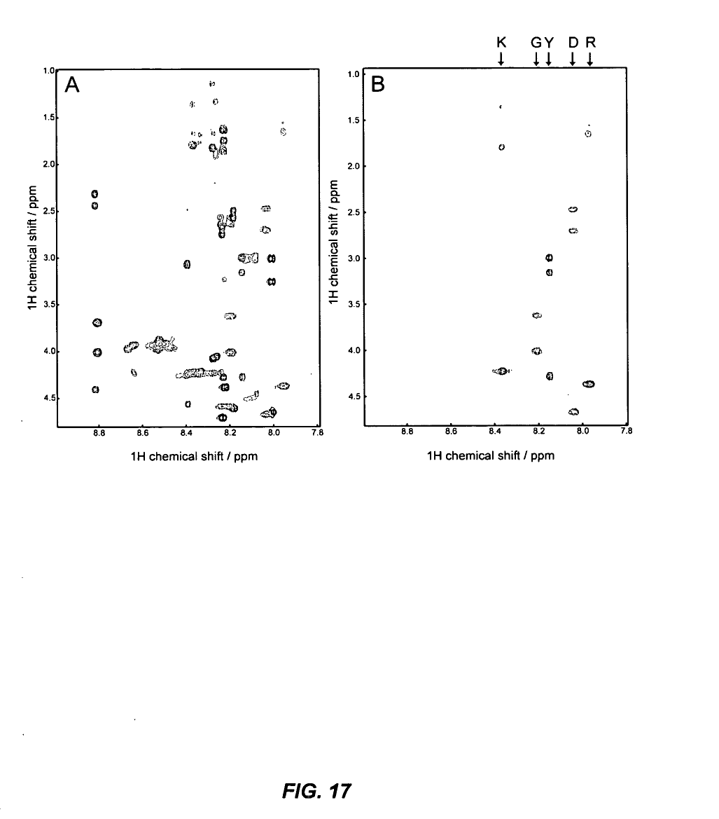

However, structural indications were observed for B7 and C7 from the NMR data. The TOCSY fingerprint regions of B7 and C7 are shown in FIG. 17. The presence of at least 20 vertical strips in the TOCSY fingerprint region of B7 (FIG. 17A) indicates that the cyclic peptide exists in a number of conformations at 10° C. This is not the result of a mixture of cyclic and linear RGDY(OMe)K peptides (i.e., a mixture of A7 and B7), because the TOCSY fingerprint region shows that two vertical strips are not present for each amino acid residue, as would be expected from a mixture of cyclic and linear peptides. After counting and assigning the vertical strips, the amino acids D and K had five or more visible conformations; Y had three conformations; G had two conformations; and R had one conformation. Analysis of the NOESY data indicates that B7 undergoes a hinge-like motion where R and G are more rigid but the DYK are capable of accessing more conformational space. Further, the NOESY data indicates that one conformation has multiple NOESY contacts, suggesting that this particular conformer is at least locked into a structural arrangement for a short period of time.

FIG. 17B shows the TOCSY fingerprint region of C7. Considering that the only difference between B7 and C7 is the addition of the 4-[¹⁹F]-fluorobenzoyl moiety to the ε-amino group of K in C7, the TOCSY NMR spectra are strikingly different. Remarkably, C7 adopts a single conformation whereas B7 adopts multiple conformations. In fact, the single conformation adopted by C7 can be observed within B7 as one of the multiple conformations of B7. Further, when the Y (L-tyrosine) in C7 was replaced with y (D-tyrosine), the peptide adopted several conformations. These results indicate that the C7 peptide structure becomes locked in a fixed single conformation when a tyrosine residue is adjacent to a lysine residue having a benzoyl moiety attached to its ε-amino group and when the tyrosine and lysine residues have the same configuration.

Without being bound to any particular theory, the remarkable ability of C7 to adopt a single conformation is provided by a pi-pi stacking interaction between the benzoyl moiety conjugated to lysine and the aromatic side chain of tyrosine. As a result, the pi-pi stacking interaction restricts (i.e., locks) C7 in a single conformation, thereby increasing its affinity and selectively for αᵥβ₃ integrin. In particular, this structural locking mechanism appears to lock the RGD sequence in a kinked structure, which has been shown to be the conformation more favorable to binding αᵥβ₃ integrin (Aumailley et al., FEBS Lett., 291:50-54 (1991)). As such, C7 is suitable for use as an imaging agent, e.g., with a radiolabeled pi-pi stacking moiety such as a 4-[¹⁸F]-fluorobenzoyl moiety, for imaging a tumor, organ, or tissue. C7 is also suitable for use as a therapeutic agent, e.g., with a radiolabeled pi-pi stacking moiety, for treating cancer, an inflammatory disease, or an autoimmune disease. Further, C1, which displayed high selectivity for α_{IIb}β₃ integrin, is suitable for use as an imaging agent or a therapeutic agent for diseases and disorders such as deep vein thrombosis (DVT).

This structural locking mechanism can also be used to restrict the conformation of other receptor-binding motifs into a more restrained structure that binds the target receptor with increased affinity and selectivity. Examples of suitable receptor-binding motifs include, without limitation, other integrin-binding motifs, growth factor receptor-binding motifs, cytokine receptor-binding motifs, TGF-β receptor-binding motifs, TNF-α receptor-binding motifs, G-protein coupled receptor-binding motifs, and combinations thereof. As such, the conformational rigidity provided by the structural locking mechanism of the present invention produces receptor-binding cyclic peptides with improved target affinity and selectivity.

### Example 3

In vivo Analysis of RGD Peptides.

This example illustrates the use of peptide C7 for the in vivo imaging of tumors.

In vivo biodistribution studies were performed in mice using A375M, a human melanoma which expresses αᵥβ₃ integrin. MF1 nu/nu mice were given subcutaneous injections of A375M cells into the left inguinal region. Once tumors reached 4-10 mm in size, mice (n=5 mice per time point) were injected with 50 kBq of [¹⁸F]-C7 and sacrificed at 15, 30, and 60 minutes after injection. A second group of animals (n=3) was injected with 50 kBq of [¹⁸F]-C7, sacrificed at 30minutes after injection, and imaged using an ECAT 951R whole body PET scanner. For imaging analysis, C7 was labeled with the radionuclide ¹⁸F instead of the nuclide ¹⁹F to create [¹⁸F]-C7.

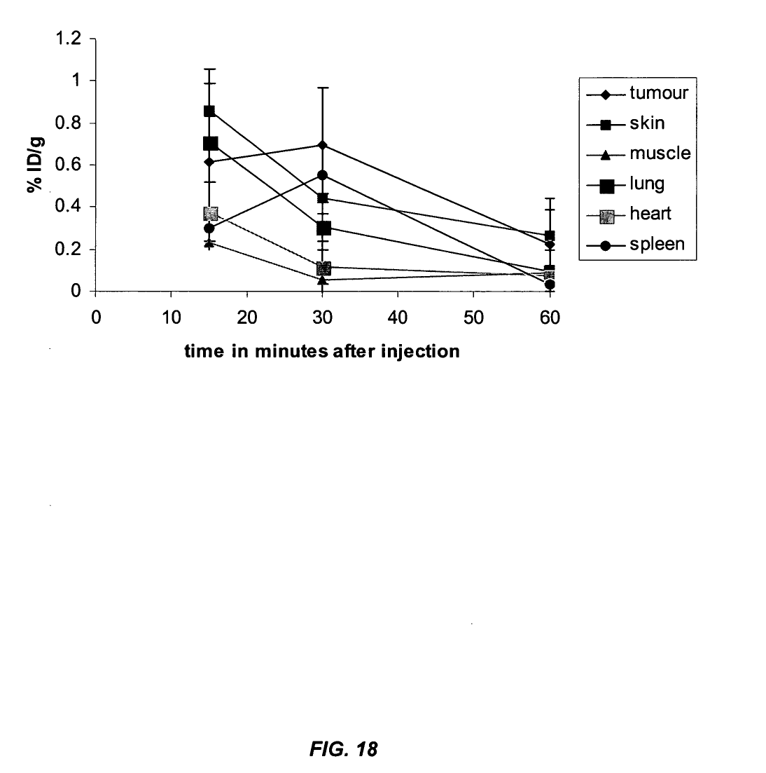

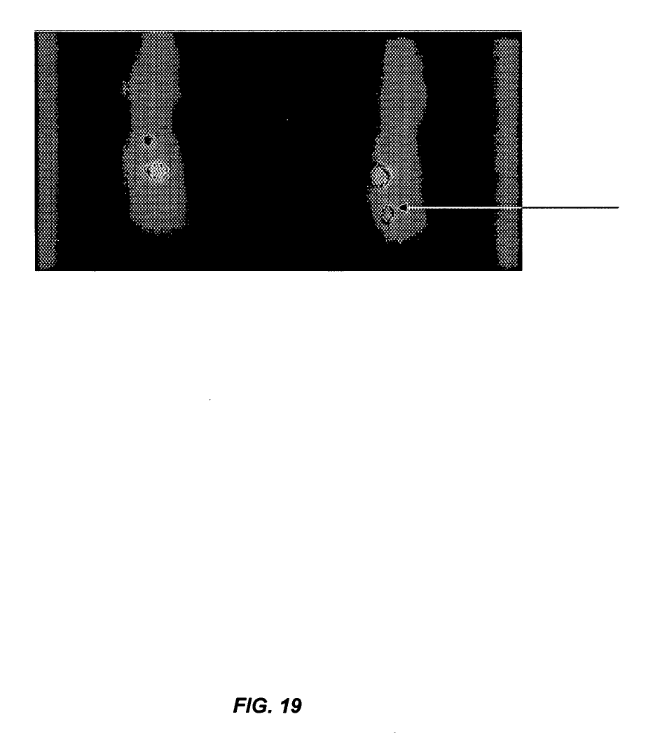

FIG. 18 shows the biodistribution of [¹⁸F]-C7 after peptide injection. At 30 minutes after injection, tumor to organ ratios of 11.67, 1.6, 2.33, and 5.83 for muscle, skin, lung, and heart, respectively were observed. The negative control peptide showed tumor to organ ratios of 1.29, 0.54, 2.15, and 1.47, respectively. Images obtained from the ECAT 951R PET scanner (FIG. 19) identified distinct areas of [¹⁸F]-C7 uptake in the lower region of the mouse (right image, arrow) that were absent in the negative control (left image).

### Example 4

Identification of αᵥβ₆-Specific Peptides.

This example illustrates the use of a molecular library approach to screen for linear and cyclic peptides that bind specifically to αᵥβ₆ integrin.

A molecular library comprising peptides having the DLXXL motif, from 0 to about 5 amino acids flanking the amino- and carboxy-termini of this motif, and the structural locking mechanism (i.e., an aromatic amino acid adjacent to a pi-pi stacking moiety conjugated to the ε-amino group of lysine) is synthesized using the one-bead-one-compound (OBOC) combinatorial library technique described in, e.g., Lam et al., Nature, 354:82-84 (1991); Lam et al., Bioorganic Medicinal Chem. Letters, 3:419-424 (1993); Lam et al., In Combinatorial Peptide and Nonpeptide Libraries—A Handbook, Gunther Jung Ed., pp. 173-201 (1996); and Lam et al., Chem. Reviews, 97:411-448 (1997).

Briefly, standard Fmoc chemistry as described in Example 1 above is used in the solid-phase synthesis of the linear and cyclic peptides of the OBOC combinatorial library. In the case of cyclic peptides, the library has cysteines at each end for disulfide cyclization or lysine or glutamate for lactam cyclization. PEG-grafted polystyrene resins are used and swollen in DMF. The resins are distributed into 19 vials and 19 of the Fmoc-protected amino acids (not cysteine) are added separately in 4-fold excess with a 4-fold excess of DIPEA and HOBt as coupling agents. Coupling is performed for about 60 minutes followed by a ninhydrin test to assess completion of the reaction. At completion, the Fmoc-protection is removed with 20% piperidine in DMF. On completion of the randomization steps, side-chain protection is removed and the peptidyl resin is washed with DMF.

To assess the importance of the DLXXL binding motif and the secondary structure of the peptides, a stepwise substitution of pairs of amino acids with the structural locking mechanism is performed. Effects of this substitution approach are investigated using a cell-based screening method. D-amino acids can then be inserted into the non-essential sites to improve in vivo stability and such peptides can be rescreened.

In vitro Screen and Optimization of OBOC Libraries

Using the “split synthesis-mix” method on solid-phase peptide synthesis, each individual peptide bead from the library displays only one peptide entity. With an appropriate detection system, the peptide bead that interacts with a specific target can be identified, isolated, and the peptide structure determined. Two screening approaches are employed in the present example:
1. In the first screen, integrin-expressing melanoma cell lines that are αᵥβ₆-negative (e.g., DX3puro) or αᵥβ₆-positive (e.g., DX3β₆puro) is used. Side-chain protecting groups are removed from the peptide beads and the beads are washed with ethanol followed by washing and suspending in DMEM. 100 μl of the bead library is incubated with DX3β₆puro cells at 37° C. for about 2 hours. Cell binding to the beads is monitored over this period. Cells that bind within the first hour are picked manually. After treatment with 1 M guanidine hydrochloride, the selected beads are then incubated with DX3puro cells. Cells that bind during this incubation period are picked out as non-specific binders, i.e., they bind both αᵥβ₃ and αᵥβ₆. The remaining αᵥβ₆-specific beads are sequenced using Edman degradation.
2. In the second screen, the peptides that bind both αᵥβ₃ and αᵥβ₆ are re-synthesized and screened in ELISA and cell-based assays as described above. Peptides that bind specifically to immobilized αᵥβ₆ (e.g., have at least 100 fold higher affinity for αᵥβ₆) are analyzed using in vivo imaging techniques.
   Serum Stability Studies

Prior to imaging, the identified peptides are incubated at 37° C. in human serum or plasma to assess their in vivo stability. Samples are taken at about 1, 2, 3, 4, 5, and 6 hour time points followed by precipitation with acetonitrile and centriftigation at 10,000×g for 1 minute. The crude sample is then analyzed using RP-TLC and RP-HPLC with on-line radioactivity and UV detection. All peaks are collected and lyophilized for mass spectrometry analysis.

Toxicity Studies

Varying concentrations of the identified peptides are incubated with cells to assess cellular toxicity. Cell viability is assessed by trypan blue staining and the colorimetric 3-(4,5-dimethyl-thiazol-2-yl)-2,5-diphenyl tetrazolium bromide (MTT) reduction assay.

The peptides identified by the above-described approach have the following characteristics: (1) low IC₅₀ (e.g., <100 nM); (2) selectivity for αᵥβ₆ (e.g., 100 fold more selective); (3) inhibition of adhesion (e.g., at <10 μM); (4) stability in serum (e.g., for at least about 2 hours); (5) non-immunogenic; and (6) non-toxic.

### Example 5

In vivo Analysis of αᵥβ₆-Specific Peptides.

This example illustrates the use of the peptides identified in Example 4 for the in vivo imaging of tumors. The peptides are radiolabeled with a 4-[¹⁸F]-fluorobenzoyl group and analyzed using microPET U1. Alternatively, the peptides can be radiolabeled with an N-succinimidyl-4-[¹²⁵I] -iodobenzoyl group.

To determine the degree of specificity of the peptides for αᵥβ₆-positive versus αᵥβ₆-negative tumors, mice bearing both DX3β₆puro αᵥβ₆-positive) and DX3puro αᵥβ₆-negative) tumors are imaged. For example, a subcutaneous injection of about 2×10⁶ DX3β₆puro and DX3puro cells is given to the opposite flanks of individual nu/nu nude mice. When tumors reach about 4 mm to about 5 mm, the mice are injected intravenously with the radiolabeled peptide and imaged for about 2 hours.

PET scanning with a microPET II small animal scanner is used for imaging the tumors in the mice. This high-resolution system produces reconstructed images with a spatial resolution of about 1.2 nm using conventional analytic reconstruction algorithms. The resolution is quite isotropic at the center of the field of view, resulting in a resolution volume of 1.7 mg of tissue. The absolute sensitivity of the scanner at the center of the field of view is 2.25% using the default energy window settings of 250-750 keV and a coincidence timing window of 10 ns. The imaging field of view of the scanner is 10 cm in the transverse direction and 4.8 cm in the axial direction. The bed is computer controlled, allowing whole-body mouse imaging to be performed in two overlapping bed positions. High quality images in mice are generally obtained using injected doses of about 50 to about 200 μCi and imaging times of about 5 to about 10 minutes.

Athymic nude mice are anesthetized with isoflurane for the duration of the imaging study. Induction of anesthesia is achieved in an induction chamber with an isoflurane concentration of 2-3%. Anesthesia is then maintained using an isoflurane concentration of 1.5-2.5% delivered through a nose cone. Radiolabeled peptide is injected as a bolus of 200 μCi into the tail vein of the mouse. A heating lamp and/or warm water can be used to dilate the tail vein to assist in peptide injection. The activity in the syringe before and after injection is measured in a dose calibrator and corrected for decay so that the injected dose is known. The mouse is positioned on a custom-built bed in the microPET II scanner. The bed has an attachment that delivers anesthesia to the mouse and is heated by recirculating warm water to maintain body temperature, which is monitored using a rectal probe. At the moment of radiolabeled peptide injection, data acquisition is initiated in the list mode on the microPET II scanner. Imaging can continue for a total of about 120 minutes. At the end of the study, the list mode data can be binned into time frames as follows: 20 frames of 60 seconds; 20 frames of 120 seconds; and 12 frames of 300 seconds. Each frame can be reconstructed with a validated statistical 3D reconstruction algorithm (see, e.g., Qi et al., Physics in Medicine and Biology, 43:1001-1013 (1998); Chatziioannou et al., IEEE Trans. Med. Imag., 19:507-512 (2000)).

Corrections for detector normalization, random coincidences, dead time, and radionuclide decay can be applied. Absolute quantification is achieved by calibrating the mouse images with the image of a cylinder containing a uniform concentration of positron-emitting radionuclide with approximately the same geometry and volume as a mouse (e.g., 2.5 cm diameter by 6 cm long=29.5 cc). The calibration scan is acquired under identical conditions and reconstruction parameters as the mouse scans and has similar attenuation and scatter characteristics. Image analysis can be carried out using ASIPro software. For each different peptide, five mice with tumor sizes in the range of about 50 to about 500 mg are imaged using microPET II to define the average pharmacokinetics of the peptide and provide information on the range and variability of the spatial and temporal distribution between mice.

In certain instances, a carrier such as octreotide can be used to improve tumor uptake of the radiolabeled peptide. Several concentrations of non-radiolabeled (i.e., cold) peptide can be titrated with radiolabeled peptide to achieve optimum dosing and the highest tumor to background ratio. Once a dosing regimen is established, blocking studies can be performed to assess specific versus non-specific binding by blocking tumor uptake with the addition of elevated doses of non-radiolabeled peptide or by injecting a non-specific radiolabeled peptide sequence such as-a scrambled DLXXL motif.

Organ Distribution of Radiolabeled Peptides

In parallel to the microPET II imaging study, 5 mice can be sacrificed at 5 time points to perform biodistribution studies and confirm the data provided by scanning. The mice can have their major organs removed, washed, and associated radioactivity determined in a Wallac gamma counter. Results can be expressed as % injected dose/g tissue. Once a correlation has been established for the tissue distribution of radiolabeled peptides versus microPET II data, biodistribution studies can be terminated. Tumors can be taken and prepared for quantitative autoradiographic imaging of the radiolabeled peptide distribution in the tumor. Blood and urine samples can also be analyzed for metabolites using RP-HPLC with on-line radioactivity detection.

Cellular and Tissue Distribution of Radiolabeled Peptides

The in vivo imaging described above can be complemented with ex vivo assays. For example, at time points corresponding to maximal tumor uptake, tumors from 3 mice can be excised, frozen, and serial sections taken onto slides. The slides are exposed to a phosphorimager and stored digitally on a computer. Samples can then be washed in TBS (pH 7.2) and incubated with 5 μg/ml αᵥβ₆-specific antibody (e.g., 10D5, a human αᵥβ₆-specific mouse monoclonal antibody available from Chemicon International) for about 1 hour to detect DX3β₆puro tumor cells. After washing, bound antibody can be detected with Alexa-488-conjugated anti-mouse IgG (Molecular Probes). Fluorescent images are collected on a typhoon autoradiography system. The autoradiographic and fluorescent digital images can then be overlaid to determine the cellular distribution of radioactivity relative to αᵥβ₆ expression.

In some embodiments, the radiolabeled peptides of the present invention, when used as in vivo molecular imaging probes, do not exhibit non-specific binding and have desired pharmacokinetic properties, e.g., renal clearance rather than hepatobiliary clearance. In certain instances, PEGylated multimers of the radiolabeled peptides are used to further improve receptor affinity and peptide clearance. In certain other instances, a PEG bridge between the pi-pi stacking moiety and the peptide is used to keep the peptide in the blood circulation for a longer period of time.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A cyclic peptide having the formula: 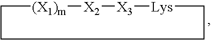
wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration.

2. A cyclic peptide according to claim 1, wherein m is 0 or 1.

3. A cyclic peptide according to claim 1, wherein said pi-pi stacking moiety is selected from the group consisting of a benzoyl group, a benzyl group, a naphthoyl group, and a naphthyl group.

4. A cyclic peptide according to claim 3, wherein said pi-pi stacking moiety is labeled with a nuclide.

5. A cyclic peptide according to claim 4, wherein said nuclide is a radionuclide.

6. A cyclic peptide according to claim 5, wherein said radionuclide is selected from the group consisting of ¹¹C, ¹³N, ¹⁵O, ¹⁸F, ⁶¹Cu, ⁶²Cu, ⁶⁴Cu, ⁶⁷Cu, ⁶⁸Ga, ¹²⁴I, ¹²⁵I, ¹³¹I.

7. A cyclic peptide according to claim 1, wherein said aromatic amino acid is selected from the group consisting of tyrosine (Tyr), phenylalanine (Phe), tryptophan (Trp), and an analog thereof.

8. A cyclic peptide according to claim 7, wherein said Tyr analog is selected from the group consisting of O-methyltyrosine (Tyr(Me)), O-benzyltyrosine (Tyr(Bzl)), homotyrosine (HoTyr), a C₁-C₄ alkyltyrosine, a C₁-C₄ alkoxytyrosine, a halotyrosine, a C₁-C₄ haloalkyltyrosine, an azidotyrosine, an aminotyrosine, a nitrotyrosine, a cyanotyrosine, a benzoyltyrosine, and a carboxytyrosine.

9. A cyclic peptide according to claim 7, wherein said Phe analog is selected from the group consisting of phenylglycine (Phg), homophenylalanine (HoPhe), a diphenylalanine, a C₁-C₄ alkylphenylalanine, a C₁-C₄ alkoxyphenylalanine, a halophenylalanine, a C₁-C₄ haloalkylphenylalanine, an azidophenylalanine, an aminophenylalanine, a nitrophenylalanine, a cyanophenylalanine, a benzoylphenylalanine, a carboxyphenylalanine, and a halophenylglycine.

10. A cyclic peptide according to claim 1, wherein said receptor-binding motif is selected from the group consisting of an integrin-binding motif, a growth factor receptor-binding motif, a cytokine receptor-binding motif, a transforming growth factor (TGF) receptor-binding motif, a tumor necrosis factor (TNF) receptor-binding motif, a G-protein coupled receptor-binding motif, a scavenger receptor-binding motif, a lipoprotein receptor-binding motif, and combinations thereof.

11. A cyclic peptide according to claim 1, wherein X₃ and Lys have an L-configuration.

12. A cyclic peptide according to claim 1, wherein said cyclic peptide adopts a single conformation.

13. A cyclic peptide according to claim 1, wherein X₂ is an integrin-binding motif; X₃ is Tyr, Tyr(Me), or Phe; the 6-amino group of Lys has a benzoyl group conjugated thereto; and X₃ and Lys have an L-configuration.

14. A cyclic peptide according to claim 13, wherein said integrin-binding motif has the amino acid sequence Arg-Gly-Asp (RGD).

15. A cyclic peptide according to claim 13, wherein said integrin-binding motif has the amino acid sequence Asp-Leu-X-X-Leu (DLXXL), and wherein X is any amino acid.

16. A cyclic peptide according to claim 13, wherein said benzoyl group is labeled with a nuclide.

17. A cyclic peptide according to claim 16, wherein said nuclide is ¹⁹F.

18. A cyclic peptide according to claim 16, wherein said nuclide is a radionuclide.

19. A cyclic peptide according to claim 18, wherein said radionuclide is selected from the group consisting of ¹⁸F, ⁶⁴Cu, and ⁶⁷Cu.

20. A cyclic peptide according to claim 13, wherein said cyclic peptide has the formula: 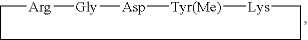
wherein
the ε-amino group of Lys has a 4-[¹⁸F]-fluorobenzoyl group or a 4-[¹⁹F]-fluorobenzoyl group conjugated thereto.

21. A cyclic peptide according to claim 20, wherein said cyclic peptide has increased selectivity for αᵥβ₃ integrin.

22. A cyclic peptide according to claim 20, wherein said cyclic peptide has increased binding affinity for αᵥβ₃ integrin.

23. A method for imaging a tumor, organ, or tissue, said method comprising:
(a) administering to a subject in need of such imaging, a cyclic peptide having the formula: 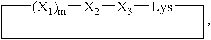
wherein
X₁ comprises m independently selected amino acids, wherein m is an integer of from 0 to 10;
X₂ is a receptor-binding motif comprising n independently selected amino acids, wherein n is an integer of from 2 to 25;
X₃ is an aromatic amino acid;
the ε-amino group of Lys has a pi-pi stacking moiety conjugated thereto; and
X₃ and Lys have the same configuration; and
(b) detecting said cyclic peptide to determine where said cyclic peptide is concentrated in said subject.

24. A method according to claim 23, wherein m is 0 or 1.

25. A method according to claim 23, wherein said pi-pi stacking moiety is selected from the group consisting of a benzoyl group, a benzyl group, a naphthoyl group, and a naphthyl group.

26. A method according to claim 23, wherein said pi-pi stacking moiety is labeled with a nuclide.

27. A method according to claim 26, wherein said nuclide is a radionuclide.

28. A method according to claim 27, wherein said radionuclide is selected from the group consisting of ¹¹C, ¹³N, ¹⁵O, ¹⁸F, ⁶¹Cu, ⁶²Cu, ⁶⁴Cu, ⁶⁸Ga, ¹²⁴I, and ¹³¹I.

29. A method according to claim 23, wherein said cyclic peptide is detected by positron emission tomography (PET).

30. A method according to claim 23, wherein said cyclic peptide is detected by Single Photon Emission Computerized Tomography (SPECT).

31. A method according to claim 23, wherein said aromatic amino acid is selected from the group consisting of tyrosine (Tyr), phenylalanine (Phe), tryptophan (Trp), and an analog thereof.

32. A method according to claim 23, wherein said receptor-binding motif is selected from the group consisting of an integrin-binding motif, a growth factor receptor-binding motif, a cytokine receptor-binding motif, a transforming growth factor (TGF) receptor-binding motif, a tumor necrosis factor (TNF) receptor-binding motif, a G-protein coupled receptor-binding motif, a scavenger receptor-binding motif, a lipoprotein receptor-binding motif, and combinations thereof.

33. A method according to claim 23, wherein X₃ and Lys have an L-configuration.

34. A method according to claim 23, wherein said cyclic peptide adopts a single conformation.

35. A method according to claim 23, wherein X₂ is an integrin-binding motif; X₃ is Tyr, Tyr(Me), or Phe; the ε-amino group of Lys has a benzoyl group conjugated thereto; and X₃ and Lys have an L-configuration.

36. A method according to claim 35, wherein said integrin-binding motif has the amino acid sequence Arg-Gly-Asp (RGD).

37. A method according to claim 35, wherein said integrin-binding motif has the amino acid sequence Asp-Leu-X-X-Leu (DLXXL), and wherein X is any amino acid.

38. A method according to claim 35, wherein said benzoyl group is labeled with a radionuclide.

39. A method according to claim 38, wherein said radionuclide is selected from the group consisting of ¹⁸F and ⁶⁴Cu.

40. A method according to claim 35, wherein said cyclic peptide has the formula: 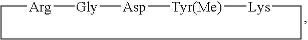
wherein
the ε-amino group of Lys has a 4-[¹⁸F]-fluorobenzoyl group conjugated thereto.

41-91. (canceled)
